(19) United States
(12) Patent Application Publication
(10) Pub. No.: US 2021/0073352 A9
(43) Pub. Date: Mar. 11, 2021

(54) SYSTEM AND METHOD FOR DRUG TARGET AND BIOMARKER DISCOVERY AND DIAGNOSIS USING A MULTIDIMENSIONAL MULTISCALE MODULE MAP

(71) Applicant: Data4Cure, Inc.

(72) Inventors: Janusz Dutkowski, San Diego, CA (US); Boguslaw Kluge, Warsaw (PL)

(21) Appl. No.: 15/545,668

(22) PCT Filed: Jan. 21, 2016
(86) PCT No.: PCT/US2016/014359

§ 371 Date: Jul. 21, 2017

Related U.S. Application Data
(60) Provisional application No. 62/107,258, filed on Jan. 23, 2015.

(51) Int. Cl. G06F 19/28; G06F 19/12

(52) U.S. Cl. CPC G06F 19/28; G06F 19/12

(57) ABSTRACT

A new method and system can be implemented to identify, analyze and display hierarchies of condition-specific gene, network or pathway activities or aberrations. Methods are also presented related to biomarker and drug-target identification and diagnosing new patients or samples with diseases or disease subtypes. Further, methods are presented related to predicting patient survival or response to treatment. Finally, methods are presented that can provide information of biological agricultural or medical interest. Methods provided herein include methods of making a multidimensional multiscale module map for identifying, analyzing and displaying hierarchies of network or pathway activities, the multidimensional multiscale map, and systems for discussing genomic features of a subject or sample with the multiscale module map.

### INCORPORATION BY REFERENCE TO ANY PRIORITY APPLICATIONS

The present application claims the benefit of priority to U.S. Provisional Patent Application No. 62/107,258, filed Jan. 23, 2015. The entire disclosure of the aforementioned application is expressly incorporated by reference in its entirety.

### BACKGROUND OF THE INVENTION

### Field of the Invention

Many human diseases can be caused by or are associated with alterations in molecular pathways, networks and processes. A new method and system can be implemented to identify, analyze and display hierarchies of condition-specific gene, network or pathway activities or aberrations. Methods are also presented that are related to biomarker and drug-target identification and diagnosing new patients or samples with diseases or disease subtypes. Further, methods are presented related to predicting patient survival or response to treatment. Presented herein are methods that can allow one or more users to identify, comment and share information of biological or clinical interest. Finally, methods are presented that can provide information of biological, agricultural or medical interest.

### Background

Instruments such as DNA and RNA sequencers, mass spectrometers, advanced imaging devices and others provide the means to characterize human diseases at the genomic and molecular level. These instruments generate a very large number of data points for an individual patient or sample. With efforts such as The Cancer Genome Atlas (TCGA) and the International Cancer Genome Consortium (ICGC), such datasets from cancer patients are accumulating at a rapid pace (Wilkinson, J. et al. Nature 455, 1061-1068 (2008), Cancer Genome Atlas Pan-Cancer/analysis project. Nat. Genet. 45, 1113-1120 (2013), and Hudson, T. J. et al. Nature 464, 993-998 (2010); all hereby incorporated by reference in their entirety herein). Similar initiatives are underway for other disease and health conditions and for healthy individuals. Extracting actionable insights from such datasets and using them to identify new biomarkers and drug target candidates is a significant challenge using available information technologies. Innovative new methods and tools for the analysis, predictive modeling and visualization of such molecular and clinical data are urgently needed.

Among the many difficulties associated with analyzing molecular datasets is dealing with biological complexity. Many diseases are highly complex at the genomic or molecular level. For example, diseases can be caused or contributed to, for example, by more than one and in some cases, many genes or proteins. Furthermore, the genetic and molecular contributors can be significantly different for different subjects or patients. For example, in cancer, while recent studies have been able to map selected driver genes based on their high mutation frequency, genes mutated frequently across many patients account for only a small fraction of all causal alterations. Furthermore, clinically relevant aberrations are not limited to the DNA sequence alone but can occur and manifest themselves at multiple molecular dimensions including the genome, transcriptome, proteome and metabolome.

Importantly, disease-associated aberrations essential to disease development, progression and outcome are not randomly distributed but organized into networks, pathways, and high-level processes. The prospect of stringing together multiple alterations and recognizing how they organize into common networks and pathways represents a significant opportunity as well as a great challenge. Recently, progress has been made in the development of pathway databases and algorithms for mining biological networks and pathways (Croft, D. et al. Nucleic Acids Res. 39, D691-D697 (2011), Ashburner, M. et al. Nat. Genet. 25, 25-29 (2000), Cerami, E. G. et al. Nucleic Acids Res. 39, D685-D690 (2011), Chuang et al. Mol. Syst. Biol. 3, 140 (2007), Kipps, T. J. et al. Blood 120, 2639-2649 (2012), Dutkowski, J et al. Protein Networks as Logic Functions in Development and Cancer. PLoS Comput. Biol. 7, e1002180 (2011), Vandin, F. et al. Genome Res. (2011), and Vaske, C. J. et al. Bioinformatics 26, (2010); all hereby incorporated by reference in their entirety herein). However, these methods typically identify disease-associated networks and pathways as flat list of distinct entities without considering their relationships to one another, in particular their hierarchical relations. This is done either a piori through the prior definition of pathways, or as part of the network search procedure. Such approaches typically produce a set of networks or pathways that are either unrelated or whose relation to each other is not clearly defined. Pathways or networks that are even slightly different (e.g. differ by one or more gene or protein members) can be considered as separate entities. Often a large number of such pathways or networks are identified that can overlap with one another posing a challenge for interpretation. A different approach can be achieved by recognizing that these networks and pathways are naturally part of a hierarchy of biological modules with specific modules being part of or a specification of larger more general module. Such a hierarchical view provides a means to capture the intricate complexity and biological activity of individual modules while at the same time capturing their higher-level relation with other modules in the system. While static databases are useful for defining the reference components, they do not capture the majority of biological contexts nor can they capture patient-specific biology. Methods and systems are needed to dynamically analyze, model and visualize multi-scale biological systems and compare system states across multiple biological or experimental conditions. Such methods and systems are needed both to advance biomedical research and development, as well as to implement genomic medicine in a clinical setting, including genome-base diagnosis and treatment.

Importantly, there is a lack of systems that would dynamically apply a large number of biomarkers and other associations extracted from data to provide direct information that can be applied towards patient diagnosis and patient-driven clinical research.

### SUMMARY OF THE INVENTION

In a first aspect, a method of making a multidimensional multiscale module map for identifying analyzing and displaying hierarchies of network or pathway activities is provided, wherein the method comprises providing a static multiscale module map, accessing a plurality of measured attributes for a plurality of elements from one or more patients and/or biological samples from at least one condition of interest, assigning a plurality of attributes to a plurality of modules in the static multiscale module map, identifying associations from a plurality of attributes for a plurality of modules and storing a database of the most significant associations along with module attributes and the static multiscale module map, thereby generating said multidimensional multiscale module map. In some embodiments, the mapping engine is coupled to the static multiscale module map. In some embodiments, the identifying is performed by an inference engine. In some embodiments, the static multiscale module map contains biological processes pertaining to human biology. In some embodiments, the biological process is a mitotic cell cycle, a gene expression pathway, a metabolic pathway, an immune system process, an adaptive immune system process, a GPCR signaling pathway, and/or a signal transduction pathway. In some embodiments, the static multiscale module map is constructed by manual curation of biological processes and pathways from literature. In some embodiments, the static multiscale module map is constructed from pathways and processes from biological databases. In some embodiments, the biological databases are Pathway Commons, Gene Ontology, and/or Reactome databases. In some embodiments, the static multiscale module map is constructed by automatic analysis of molecular networks to infer underlying hierarchical structure. In some embodiments, the static multiscale module map is inferred from biological data. In some embodiments, the biological data includes molecular interaction data, protein-protein interaction data, genetic data, transcriptional data, protein-DNA interaction data, and/or a combination thereof. In some embodiments, the biological data represents at least one group of genes and/or proteins that correspond to a biological process. In some embodiments, the static multiscale module map is inferred from iterative findings of cliques in a molecular interaction network. In some embodiments, the static multiscale module map is constructed from manually curated databases arranged into hierarchies by exploiting the hierarchical relationships from input biological databases or by identifying pairs of modules for at least one set of genes of at least one module. In some embodiments, the plurality of measured attributes for a plurality of elements from one or more patients comprises omics data from patients and/or omics data from cell lines. In some embodiments, the omics data comprises data from genomics, cognitive genomics, functional genomics, metagenomics, epigenomics, lipidomics, proteomics, immunoproteomics, proteogenomics, structural genomics, transcriptomics, pharmacogenomics, toxicogenomics, stem cell genomics and/or metabolomics. In some embodiments, the multidimensional multiscale module map comprises an integrated network of patient genomic data with the static multiscale modular map to identify common modules and pathways altered in a disease. In some embodiments, the genomic data comprises somatic mutations, gene copy-number, gene expression data, somatic mutations, gene copy-number, DNA sequences, RNA sequences, and/or proteomic measurements. In some embodiments, the multidimensional multiscale module map comprises modules. In some embodiments, the modules represent genes, proteins, mRNA, microRNA, amino acid sequences, DNA sequences, protein complexes, cellular components, functions or processes or other biological entities and/or a combination thereof. In some embodiments, the multidimensional multiscale module map comprises associations between modules. In some embodiments, the associations between modules comprises biological and clinical characteristics. In some embodiments, the multidimensional multiscale module map further comprises associations between modules and biological phenotypes and/or clinical phenotypes, thereby generating inferred associations on the multidimensional multiscale module map. In some embodiments, the biological and clinical characteristics comprises survival predictions, response to treatment predictions, health conditions, disease types, disease subtypes and/or other traits associated with patients or biological samples. In some embodiments, the patient suffers from cancer. In some embodiments, the cancer is lung adenocarcinoma, lung squamous cell carcinoma, lung cancer, lung squamous cell carcinoma, lung cancer, breast cancer, kidney cancer, or leukemia. In some embodiments, the multidimensional multiscale module map comprises at least one identifier for at least one condition of interest. In some embodiments, the at least one condition of interest is a disease and/or cancer. In some embodiments, the cancer is lung adenocarcinoma, lung squamous cell carcinoma, lung cancer, lung squamous cell carcinoma, lung cancer, breast cancer, kidney cancer, or leukemia.

In a second aspect a multidimensional multiscale module map for identifying analyzing and displaying hierarchies of network or pathway activities, generated by any one or any combination of embodiments described herein. In some embodiments, the method of making a multidimensional multiscale module map for identifying analyzing and displaying hierarchies of network or pathway activities is provided, wherein the method comprises providing a static multiscale module map, accessing a plurality of measured attributes for a plurality of elements from one or more patients and/or biological samples from at least one condition of interest, assigning a plurality of attributes to a plurality of modules in the static multiscale module map, identifying associations from a plurality of attributes for a plurality of modules and storing a database of the most significant associations along with module attributes and the static multiscale module map, thereby generating said multidimensional multiscale module map. In some embodiments, the mapping engine is coupled to the static multiscale module map. In some embodiments, the identifying is performed by an inference engine. In some embodiments, the static multiscale module map contains biological processes pertaining to human biology. In some embodiments, the biological process is a mitotic cell cycle, a gene expression pathway, a metabolic pathway, an immune system process, an adaptive immune system process, a GPCR signaling pathway, and/or a signal transduction pathway. In some embodiments, the static multiscale module map is constructed by manual curation of biological processes and pathways from literature. In some embodiments, the static multiscale module map is constructed from pathways and processes from biological databases. In some embodiments, the biological databases are Pathway Commons, Gene Ontology, and/or Reactome databases. In some embodiments, the static multiscale module map is constructed by automatic analysis of molecular networks to infer underlying hierarchical structure. In some embodiments, the static multiscale module map is inferred from biological data. In some embodiments, the biological data includes molecular interaction data, protein-protein interaction data, genetic data, transcriptional data, protein-DNA interaction data, and/or a combination thereof. In some embodiments, the biological data represents at least one group of genes and/or proteins that correspond to a biological process. In some embodiments, the static multiscale module map is inferred from iterative findings of cliques in a molecular interaction network. In some embodiments, the static multiscale module map is constructed from manually curated databases arranged into hierarchies by exploiting the hierarchical relationships from input biological databases or by identifying pairs of modules for at least one set of genes of at least one module. In some embodiments, the plurality of measured attributes for a plurality of elements from one or more patients comprises omics data from patients and/or omics data from cell lines. In some embodiments, the omics data comprises data from genomics, cognitive genomics, functional genomics, metagenomics, epigenomics, lipidomics, proteomics, immunoproteomics, proteogenomics, structural genomics, transcriptomics, pharmacogenomics, toxicogenomics, stem cell genomics and/or metabolomics. In some embodiments, the multidimensional multiscale module map comprises an integrated network of patient genomic data with the static multiscale modular map to identify common modules and pathways altered in a disease. In some embodiments, the genomic data comprises somatic mutations, gene copy-number, gene expression data, somatic mutations, gene copy-number, DNA sequences, RNA sequences, and/or proteomic measurements. In some embodiments, the multidimensional multiscale module map comprises modules. In some embodiments, the modules represent genes, proteins, mRNA, microRNA, amino acid sequences, DNA sequences, protein complexes, cellular components, functions or processes or other biological entities and/or a combination thereof. In some embodiments, the multidimensional multiscale module map comprises associations between modules. In some embodiments, the associations between modules comprises biological and clinical characteristics. In some embodiments, the multidimensional multiscale module map further comprises associations between modules and biological phenotypes and/or clinical phenotypes, thereby generating inferred associations on the multidimensional multiscale module map. In some embodiments, the biological and clinical characteristics comprises survival predictions, response to treatment predictions, health conditions, disease types, disease subtypes and/or other traits associated with patients or biological samples. In some embodiments, the patient suffers from cancer. In some embodiments, the cancer is lung adenocarcinoma, lung squamous cell carcinoma, lung cancer, lung squamous cell carcinoma, lung cancer, breast cancer, kidney cancer, or leukemia. In some embodiments, the multidimensional multiscale module map comprises at least one identifier for at least one condition of interest. In some embodiments, the at least one condition of interest is a disease and/or cancer. In some embodiments, the cancer is lung adenocarcinoma, lung squamous cell carcinoma, lung cancer, lung squamous cell carcinoma, lung cancer, breast cancer, kidney cancer, or leukemia.

In a third aspect, a method for graphically displaying information and data from a generated multidimensional multiscale module map is provided, wherein the method comprises providing the generated multidimensional multiscale module map for identifying analyzing and displaying hierarchies of network or pathway activities of any of the embodiments described herein, generating a color map from the generated multidimensional multiscale module map and displaying the color map on a screen of a computer device or system. In some embodiments, the method further comprises displaying modules of interest. In some embodiments, the method further comprises displaying hierarchical relationships between modules. In some embodiments, the method further comprises displaying associations between module characteristics and between modules and phenotype characteristics.

In a fourth aspect, a method for identifying multiscale biomarkers of a biological or medical condition of interest is provided, wherein the method comprises providing the generated multidimensional multiscale module map of any of the embodiments described herein for identifying analyzing and displaying hierarchies of network or pathway activities, selecting at least two conditions from the multidimensional multiscale module map, wherein at least one of the conditions is a condition of interest and another condition is a reference condition and querying the multidimensional multiscale module map for significant associations for the condition of interest using a query system. In some embodiments, the condition is a disease or cancer. In some embodiments, the cancer is lung adenocarcinoma, lung squamous cell carcinoma, lung cancer, breast cancer, kidney cancer, or leukemia.

In a fifth aspect, a method for identifying common multiscale biomarkers in multiple conditions of interest is provided, wherein the method comprises providing the multidimensional multiscale module map of any of the embodiments described herein, inferring the multiscale biomarkers for each condition of interest, wherein the multiscale biomarkers are identified by any one of the embodiments described herein, and querying the multidimensional multiscale module map for biomarkers common to a condition of interest using a query system. In some embodiments, the condition of interest is a disease or cancer. In some embodiments, the cancer is lung adenocarcinoma, lung squamous cell carcinoma, lung cancer, breast cancer, kidney cancer, or leukemia.

In a sixth aspect, a method for diagnosing a patient or sample is provided, wherein the method comprises providing the multidimensional multiscale module map of any of the embodiments described herein, wherein the multidimensional multiscale module map comprises inferred associations for the conditions of interest, assigning a plurality of sample attributes to a plurality of modules in the multidimensional multiscale module map, automatically querying the multidimensional multiscale module map for associations matching the one or more mapped module attributes in the query patient or biological sample and generating a list of the identified associations ranked based on significance and/or module size criteria or other user selected criteria. In some embodiments, the user selected criteria comprises significance threshold, type of association of interest, statistical test used, and/or datasets used to derive the association. In some embodiments, the assigning is performed by a mapping engine. In some embodiments, the mapping engine is coupled to a static or a multidimensional multiscale module map.

In a seventh aspect, a system for discussing genomic features of a subject or sample is provided, wherein the system comprises a set of user information embodied in a computer readable medium that represents users that wish to participate in online discussions, a set of instructions for transmitting online discussion between users, an interactive visualization framework with a web-browser interface and the multidimensional multiscale module map of any of the embodiments provided herein. In some embodiments, the multidimensional multiscale module map is obtained from a cloud based online system. In some embodiments, the system can be implemented on a single computer, a server, a cluster of computers and/or servers.

In an eight aspect, a method for providing a group forum for discussing genomic features of a subject or sample is provided, wherein the method comprises providing the system of any of the embodiments described herein and transmitting online discussions between users.

In a ninth aspect, a method for providing a group forum for discussing genomic features of a subject or sample and potential treatment options is provided, wherein the method comprises providing the system of any one of embodiments described herein and transmitting online discussions between users. In some embodiments, the genomic features are potential biomarkers. In some embodiments, the genomic features are drug targets. In some embodiments, the genomic features are matched with statistical associations stored in a database.

In a tenth aspect, a method for identifying multiscale biomarkers of a biological or medical condition of interest is provided, wherein the method comprises providing the generated multidimensional multiscale module map of any of the embodiments described herein for identifying analyzing and displaying hierarchies of network or pathway activities, selecting at least two conditions from the multidimensional multiscale module map, wherein at least one of the conditions is a condition of interest and another condition is a reference condition and querying the multidimensional multiscale module map for significant associations for the condition of interest using an inference engine and storing a database of the most significant associations, thereby generating a database of identified multiscale biomarkers. In some embodiments, the condition is a disease or cancer. In some embodiments, the cancer is lung adenocarcinoma, lung squamous cell carcinoma, lung cancer, breast cancer, kidney cancer, or leukemia.

In an eleventh aspect, a system for discussing modules and module relations in a static multiscale module map is provided, wherein the system comprises a set of user information embodied in a computer readable medium that represents users that wish to participate in online discussions, a set of instructions for transmitting online discussion between users, an interactive visualization framework with a web-browser interface and a static multiscale module map. In some embodiments, the static multiscale map is constructed by manual curation of biological processes and pathways from literature. In some embodiments, the static multiscale map is constructed from pathways and processes from biological databases. In some embodiments, the biological databases are Pathway Commons, Gene Ontology, and/or Reactome databases. In some embodiments, the static multiscale module map is constructed by automatic analysis of molecular networks to infer underlying hierarchical structure. In some embodiments, the static multiscale module map is inferred from biological data. In some embodiments, the biological data includes molecular interaction data, protein-protein interaction data, genetic data, transcriptional data, protein-DNA interaction data, and/or a combination thereof. In some embodiments, the biological data represents at least one group of genes and/or proteins that correspond to a biological process. In some embodiments, the static multiscale module map is inferred from iterative findings of cliques in a molecular interaction network. In some embodiments, the static multiscale module map is constructed from manually curated databases arranged into hierarchies by exploiting the hierarchical relationships from input biological databases or by identifying pairs of modules for at least one set of genes of at least one module. In some embodiments, the static multiscale module map is obtained from a cloud based online system. In some embodiments, the system can be implemented on a single computer, a server, a cluster of computers and/or servers.

In a twelfth aspect, a system for discussing modules, modules relations, module attributes or activities and module associations in the multidimensional map is provided, wherein the system comprises a set of user information embodied in a computer readable medium that represents users that wish to participate in online discussions, a set of instructions for transmitting online discussion between users, an interactive visualization framework with a web-browser interface and the multidimensional multiscale module map of any of the embodiments described herein. In some embodiments, the multidimensional multiscale module map is obtained from a cloud based online system. In some embodiments, the system can be implemented on a single computer, a server, a cluster of computers and/or servers.

### BRIEF DESCRIPTION OF THE DRAWINGS

FIG. 1 shows the construction of the multidimensional multi-scale map. Pathway repositories and ontologies are integrated to form a hierarchical module map. Input features describing patient omics data can be integrated and transformed into module-level attributes via a mapping engine. An inference engine can be used to infer associations between module attributes and between module attributes and clinical or biological conditions or traits. Module attribute values can be compared across multiple patients or samples that can be categorized into different classes or can have different biological or clinical characteristics such as survival, response to treatment, health condition, disease type or subtype or other traits that can be associated with patients or biological samples. An inference engine can be used that implements, for example, one or more statistical tests, for example a T-test, cox regression analysis, hypergeometric test, binomial test, gene set enrichment analysis, or another test commonly used in bioinformatics. Identified associations can be stored in a database along with the multidimensional multiscale map and queried to provide biological or clinical information on-demand. New inferences based on the stored map can also be performed based on module attributes or module and clinical or biological attributes selected by the user. ‘Mut’ indicates a gene mutation occurring in one or more of the genes in a module, ‘Expr’ indicates average gene expression for genes in a module, ‘CNV’ indicates a copy-number variant in one or more of the genes in a module. ‘˜’ indicates an inferred association between attributes or biological or clinical traits. Associations can have additional parameters that are not shown here such as, for example, a sign, P-value, Q-value, statistical score.

FIG. 2 shows examples of aggregated module-level attribute values for selected conditions or time points in the multidimensional map. Also shown are examples of statistical associations that can be associated with modules in the multidimensional multiscale map that describes multiple conditions or time points of interest.

FIG. 3 shows an example of a static multiscale map containing biological processes pertaining to human biology. The map is constructed by manual curation of biological processes and pathways from literature. Pathways and processes from the Pathway Commons and Reactome databases were considered to build this process map. Each node in the graph (filled circle) represents a biological module, e.g. a biological process, a pathway or a gene. Each edge (line connecting two nodes) represents a parent-child relation between modules, for instance, such that one module is considered a parent of another module or one module contains another module, or one module is a generalization of another module. Each module can have multiple children and multiple parents. A root in the hierarchy does not have any parents. Other nodes are labeled with the corresponding names of biological process or pathways. For display purposes, only the names of selected top-level pathways and processes are shown. Node size can indicate the number of genes or other biological entities assigned to the corresponding modules.

FIG. 4 shows an example of a static multiscale map containing biological processes pertaining to human biology. The map is constructed by automatic analysis of molecular networks in order to infer their underlying hierarchical structure. Each node in the graph (filled circle) represents a group of genes or proteins which can in turn correspond to a biological module, e.g. a biological process, a pathway or a gene. Each edge represents a parent-child relation between modules, for instance, such that one module is considered a child of another module or one module contains another module, or one module is a generalization of another module. Each module can have multiple children and multiple parents. A root in the hierarchy does not have any parents. Other nodes are labeled with the corresponding names of biological process or pathways. For display purposes, only the names of selected top-level pathways and processes are shown. Node size can indicate the number of genes or other biological entities assigned to the corresponding modules. Node sizes indicate the number of genes assigned to a module. Node colors can represent the degree of correspondence to a module in Gene Ontology as determined by ontology alignment, with high-level alignments labeled. Insets show the hierarchy identified for the ribosome and actin cytoskeleton.

FIG. 5 shows an example of a multidimensional multiscale map, where module colors indicate the change in mutation frequency of genes falling within a given module. The change is computed by comparing samples across two or more states to identify mutations that occurred in a particular state and then identifying the modules in the map which have a significantly greater mutation frequency then expected at random, given the overall mutation frequency of the given biological condition. Modules which display significantly more somatic mutations in lung adenocarcinoma samples than expected at random are shown in darker color. Somatic mutations were determined by comparing lung adenocarcinoma samples with normal samples from the same patients.

FIG. 6 shows part of the map from FIG. 4 and is focused on the module 9853 (Table 3 list the list of genes in this module), labeled as “myosin filament”. Genes underneath this module often harbor somatic mutations in lung adenocarcinomas. The bar chart associated with the selected module shows the number of study cohort patients that have mutations in the genes in this module.

FIG. 7 shows an example of a multidimensional multiscale map, where module colors indicate the change in module activity from one condition to another. The change is computed by comparing samples across two states using a statistical test such as a t-test or Wilcoxon test or another test used to compare a continuous or categorical variable across two sets of samples known to those skilled in the art. Here modules are shown in darker color if the average gene expression for genes assigned to the module is significantly different in breast cancer samples that harbor a mutation in TP53 gene than in samples with wild-type TP53 gene. Modules can be assigned different colors based on lower or higher average gene expression or another measurement that can be assigned to modules.

FIG. 8 shows part of the map from FIG. 6 and is focused on the module labeled as “Cyclin B2 mediated events”. Genes in this module have a higher average gene expression in breast cancer samples that harbor a mutation in TP53 gene (indicated as ‘1’) than in samples with wild-type TP53 (indicated as ‘0’).

FIG. 9 shows part of the map from FIG. 6 and is focused on a module labeled as “Regulation of Cytoskeletal Remodeling and cell Spreading by IPP Complex Components”. Genes in this module have higher average gene expression in breast cancer samples that harbor a mutation in TP53 gene (indicated as ‘1’) than in samples with wild-type TP53 (indicated as ‘0’).

FIG. 10 shows part of a multidimensional multiscale map, where module colors indicate the association between the event of a mutation in a gene within a given module and patient survival in Kidney Renal Clear Cell Carcinoma patients computed using Cox regression analysis. Patients who have a mutation in one of the genes in the selected module (patient group indicated as ‘True’) have shorted overall survival than patients who do not have a mutation in a gene in this module (patient group indicated as ‘False’).

FIG. 11 shows part of a multidimensional multiscale map, where module colors indicate the change in drug sensitivity depending on the mutation status of a given module (i.e. module can contain a mutation in one of the genes or not). The change is computed by comparing drug sensitivity for samples with or without mutations within genes belonging to a module. Drug sensitivity is measured by the IC 50 index. A statistical test such as a T-test or Wilcoxon test or another test known to those skilled in the art can be used to compare the IC50 scores across two sets of samples. Here mutations in genes belonging to the selected module (module number 17552) are associated with increased sensitivity to the compound AZD6244. Cell lines with a relevant mutation are indicated as “1” in the boxplot and tend to have lower IC 50 values indicating higher sensitivity to AZD6244.

FIG. 12 shows modules represented by nodes in the multidimensional multiscale map can be assigned detailed network views which can display relationships between entities belonging to the module. Here an example of a detailed view for the REACTOME Fanconi Anemia Pathway is shown.

FIG. 13 shows that multidimensional multiscale map can be implemented as part of a computer system, for instance a web-based or a cloud-based online system. Here an example of a multidimensional multiscale map is visualized using an interactive visualization framework with a web-browser interface. The interface allows the user to zoom in and out of the selected regions of the map and search for selected biological modules. The user can dynamically select which module scores for which conditions or states are visualized. The system can also provide the ability to search for modules (nodes) or module relations or associations (edges) in the network based on matching user supplied text with names of relevant modules in the map. When a search is performed, nodes or edges matching search criteria can be displayed on a list. After the user selects one of the nodes or edges in the list, the network can be focused on the selected node with appropriate zoom values to make the selected node or edge clearly visible to the user. Values assigned to modules or genes or other entities preferably appearing as nodes in the networks can be presented in a table, for example, with one row per node in the network. The table can preferably be integrated with the network to allow automatic focusing on relevant table row or a network node when the user selects a node or table row, respectively. Similarly a table can be implemented where rows preferably correspond to network edges and the table can be integrated with the network to allow automatic refocusing of the table or the network when the user selects an edge or a network row, respectively.

FIG. 14 shows a cloud-based online system with a web-browser interface for analyzing a genome sequence from a patient or sample, either the entire genome sequence of an organism or parts of the genome for example the exome of an organism. The user can also use the system to analyze, for example, the somatic variants in a tumor genome where the germline variants have been removed either prior to the analysis or during the analysis using specific filters that the invention can implement. The system displays a human reference genome or a genome of another organism. Germline or somatic variants of a subject or sample can be shown around this reference, either above, over or below the reference genome. A table of germline or somatic variants is displayed and coordinated with the genome view. The table contains variant identification information and annotations such as occurrence within general populations or disease populations and other variant annotations or scores, statistics, measurements, p-values, q-values, or other values associated with a variant. The table can be integrated with the genome view. For example, when the user selects a row or variant in the table the genome view is refocused on that variant. Conversely, if the user selects a variant in the genome view, the table is scrolled and refocused to display variant information.

FIG. 15 shows filters that allow the user to identify a subset of interesting variants based on annotations and statistics associated with the variants. The user is able to select interesting filters from a list of available filters and enable them. Enabled filters are then used by the system to subselect rows in the variant table.

FIG. 16 variants which pass specified filters can be displayed on a multidimensional multiscale map or on a static map here shown as triangles. Variants can be displayed as individual nodes. They can be connected or shown close to modules in the map which are affected by these variants. Variants and modules affected by variants can also be indicated on the map by a color that can be defined by a key.

FIG. 17 shows an association view that provides means to display and browse the list of high-scoring or significant associations of variants, genes affected by variants or modules affected by variants with other genes, modules or biological or clinical entities or their measurements. The system allows the user to visualize the data underlying the statistical association selected. Here a boxplot is presented to visualize differential gene expression.

FIG. 18 shows a discussion wall that allows users to post and discuss variants, genes, modules, relations or other biological or clinical features specific to the subject or sample. Text comments can be added with each post and users can reply with text comments. New posts can be made by clicking on a button or link which can be accessible from a row in the variant table or in the association table or in the network table. The post contains a reference to the module or entity associated with the table row and can contain an additional textual comment.

FIG. 19 shows how responses to the post can be made either from the wall or directly from the variant table or the association table or the network table from which the original post was made. The system displays in each row the information on the number of posts associated with the entity in the row and allows either placing new posts or replying to prior posts made by the same user or a different user or users.

FIG. 20 shows the various possible components of the system and their interactions. The mapping engine can take the static multiscale map and the multidimensional data from patients or samples and produce the mapped data with features describing modules in the map. The inference engine can take the mapped data and optionally also additional features describing biological or clinical attributes or conditions to infer associations that are stored with the multidimensional map. The query engine can be used either to identify associations matching a user query, or to identify associations that match the mapped features of a subject or sample. The visualization engine can be used to visualize the multidimensional multiscale map, the genomic features or the inferred associations. The visualization engine can include, among other systems, a genome visualization system, a network map visualization system and an association visualization system. The visualization engine can be coupled with a system for discussing genomic features, modules, module features and/or module associations.

### DETAILED DESCRIPTION

All patents, applications, published applications and other publications referred to herein are incorporated by reference for the referenced material and in their entireties. If a module or phrase is used herein in a way that is contrary to or otherwise inconsistent with a definition set forth in the patents, applications, published applications and other publications that are herein incorporated by reference, the use herein prevails over the definition that is incorporated herein by reference.

As used herein, the singular forms “a”, “an”, and “the” include plural references unless indicated otherwise, expressly or by context.

A “static multiscale module map” as described herein refers to a generated map that contains biological processes pertaining to human biology. The static multiscale map can be constructed by manual curation of biological processes and pathways from literature, by automatic analysis of molecular networks in order to infer their underlying hierarchical structure. The static multiscale map can comprise nodes that can represent a biological module, such as for example a biological process, a pathway and/or a gene. The static multiscale map can also be constructed by automatic analysis of molecular networks in order to infer their underlying hierarchical structure. The static multiscale map can also be inferred from data. The data can comprise biological data, such as, for example, molecular interaction data—such as protein-protein, genetic, transcriptional, protein-DNA, or other biological networks or a combination of such networks—can be performed using a method known to those skilled in the art. The static multiscale map can be constructed using a method which iteratively joins the most similar entities to construct a binary clustering tree or dendrogram. A static multiscale network map can also be inferred using a procedure which constructs a binary tree or dendrogram in a way to optimize the probability of the underlying interaction data. A binary tree can first be constructed and further refined to a non-binary tree by subsequent local optimization of the probability score. A method based on iterative finding of cliques in a network with weighted edges can be applied to construct the static multiscale module map. In some embodiments described herein, a static multiscale map is provided for making a multidimensional multiscale map. The static multiscale map can be visualized as described in Dutkowski et al. (Nucleic Acids Res 42(1): D1269-74 (2014); incorporated by reference in its entirety herein).

A “multidimensional multiscale module map” as described herein, refers to a multiscale map of data, wherein the map comprises data from patients, cell lines or animal models that are integrated and transformed into module level scores. The modules can be compared across multiple patients or samples that can be categorized into different classes or can have different biological or clinical characteristics such as survival, response to treatment, health condition, disease type or subtype or other traits that can be associated with patients or biological samples. The module level scores can be obtained by a statistical test, for example a T-test, Cox regression analysis, hypergeometric test, binomial test, gene set enrichment analysis, or another test commonly used in bioinformatics can be performed using aggregated module-level scores to compare the multiscale module scores across conditions or sample categories or to correlate them with a biological or clinical trait. Other statistical tests can be used and are known to those skilled in the art. Identified statistical associations can be stored in a database and queried to provide biological or clinical information on-demand. The statistical associations that can be associated with modules in the multidimensional multiscale map can be used to describe multiple conditions or time points of interest. The multidimensional map can be based on a static map and can contain additional data and/or information.

“Modules,” as described herein, refers to loci, protein complexes, biological networks, biological pathways, higher-level biological processes and biological functions. Modules can represent a collection of one or more genes, proteins, mRNA, microRNA, amino acid sequences, DNA sequences, protein complexes, cellular components, functions or processes or other biological entities. In some embodiments, the modules comprise one or more genes, proteins, mRNA, microRNA, amino acid sequences, DNA sequences, protein complexes, cellular components, functions or processes or other biological entities. Modules can also implement data such as for example, epigenetic, transcriptomic, proteomic alterations or other biological or clinical dimensions known to those skilled in the art. Modules can also represent genomic and transcriptomic aberrations at multiple biological scales, such as individual loci, pathways, and high level processes. In some embodiments, the modules are significant high scoring modules. In some embodiments described herein the modules and/or module associations refer to biomarkers for a specific condition. In some embodiments, the modules and/or module associations are biomarkers for patient survival. In some embodiments, the modules and/or module associations are biomarkers for patient response to treatment. In some embodiments, the modules can provide new drug targets. In some embodiments, the modules provide information of agricultural interest. The module significance and module size are determined computationally. In some embodiments described herein, the modules represent genes, proteins, mRNA, microRNA, amino acid sequences, DNA sequences, protein complexes, cellular components, functions or processes or other biological entities and/or a combination thereof. In some embodiments, the multidimensional multiscale module map comprises associations between module attributes and/or between module attributes and biological or clinical attributes. In some embodiments, the associations between modules comprises biological and clinical characteristics. In some embodiments, the biological and clinical characteristics comprises survival predictions, response to treatment predictions, health conditions, disease types, disease subtypes and/or other traits associated with patients or biological samples. In some embodiments described herein, the multidimensional module map comprises modules, wherein the modules can be visualized using conventional programming codes, software analysis and/or processing systems as provided herein.

“Biomarker” as described herein, can refer to an indicator of severity of disease state, a process, a biological process, and/or a physiological state of an organism, and are known to those skilled in the art. Biomarkers can be discovered by genomic approaches, proteomic approaches, metabolomics approaches, lipidomics approaches, and other scientific methods known to those skilled in the art. By way of example and not of limitation, they can be discovered through northern blots, gene expression, DNA microarray, 2D-PAGE gel, LC-MS, MALDI-TOF, Antibody array, tissue microarray, and other methods known to those skilled in the art. In some embodiments, a method for identifying multiscale s of a biological or medical condition of interest is provided. As described herein, several embodiments can be used to provide biomarkers that predict patient survival. Biomarkers can include but are not limited to genes, proteins, and other markers that indicate a severity of disease state, a process, a biological process, and/or a physiological state of an organism, and other indicators that are known to those skilled in the art. In some embodiments, the biomarkers are represented as modules. In some embodiments, the modules contain genes. In some embodiments, biomarkers can be used to predict sensitivity to drugs or compounds. In some embodiments, biomarkers can be used to correlate mutation to survival rates. Examples of biomarkers that are used to predict sensitivity to drugs or compounds are shown for example in Table 1. Examples of biomarkers used for correlation to survival are shown in Table 2.

In some embodiments described herein, methods and systems are provided to predict survival of kidney renal cell carcinoma cancer patients (identified as KIRC in Table 2). In some embodiments, patients which harbor mutations in any of the genes in the module 13986 can be predicted to have shorter overall survival (the list of genes in this module is provided in Table 3). In some embodiments, patients which harbor mutations in any of the genes in the module 16245 can be predicted to have shorter overall survival (the list of genes in this module is provided in Table 3). In some embodiments, patients which harbor mutations in any of the genes in the module 15406 can be predicted to have shorter overall survival (the list of genes in this module is provided in Table 3). In some embodiments, patients which harbor mutations in any of the genes in the module 17177can be predicted to have shorter overall survival (the list of genes in this module is provided in Table 3).

In some embodiments, the systems and methods described herein can be used to predict survival of head and neck cancer squamous cell carcinoma patients (identified as HNSC in Table 2) patients. In some embodiments, patients which harbor mutations in any of the genes in the module 17542 can be predicted to have shorter overall survival (the list of genes in this module is provided in Table 3). In some embodiments, patients which harbor mutations in any of the genes in the module 17364 can be predicted to have shorter overall survival (the list of genes in this module is provided in Table 3).

In some embodiments, the systems and methods described herein can be used to predict survival of lung squamous cell carcinoma cancer patients (identified as LUSC in Table 2) patients. In some embodiments patients which harbor mutations in any of the genes in the module 15022 can be predicted to have longer overall survival (the list of genes in this module is provided in Table 3).

In some embodiments of the methods, systems and multiscale maps, the methods, systems and multiscale maps can be used to provide biomarkers that predict response to treatment with certain drugs or compounds.

In some embodiments, patient or sample tumors or cancers that harbor mutations in any of the genes in module 17611 can be predicted to be more sensitive to PD.0325901, AZD6244 and MEK inhibitors (the list of genes in this module is provided in Table 3).

In some embodiments, patient or sample tumors or cancers that harbor mutations in any of the genes in module 17552 can be predicted to be more sensitive to PD.0325901, AZD6244 and MEK inhibitors (the list of genes in this module is provided in Table 3).

In some embodiments, patient or sample tumors or cancers that harbor mutations in any of the genes in module 16873 can be predicted to be more sensitive to PD.0325901, AZD6244 and MEK inhibitors (the list of genes in this module is provided in Table 3).

In some embodiments, patient or sample tumors or cancers that harbor mutations in any of the genes in module 16157 can be predicted to be more sensitive to PD.0325901, AZD6244 and MEK inhibitors (the list of genes in this module is provided in Table 3).

TABLE 1

| Mutation in module | Sensitivity to drug | T-Statistic | P-Value |
|---|---|---|---|
| 17611 | PD.0325901 | −7.110886328 | 1.15E−12 |
| 17611 | AZD6244 | −7.070508158 | 1.54E−12 |
| 17611 | MEK inhibitor | −7.049196588 | 1.80E−12 |
| 17552 | PD.0325901 | −8.773930106 | 0 |
| 17552 | AZD6244 | −8.559811577 | 0 |
| 17552 | MEK inhibitor | −8.580408885 | 0 |
| 16873 | PD.0325901 | −10.5188963 | 0 |
| 16873 | AZD6244 | −9.93684112 | 0 |
| 16873 | MEK inhibitor | −9.951390393 | 0 |
| 16157 | AZD6244 | −6.875529376 | 6.18E−12 |
| 16157 | MEK inhibitor | −6.884779629 | 5.79E−12 |
| 16157 | PD.0325901 | −6.692170719 | 2.20E−11 |

TABLE 2

| | | | | | | N subjects |
|---|---|---|---|---|---|---|
| Cancer | | Mutation | LR | Wald | SC | with |
| Type | Module | predictive of | Pvalue | Pvalue | Pvalue | mutation |
| KIRC | 13986 | Shorter | 6.78E−07 | 1.28E−07 | 3.55E−08 | 14 |
| | | survival | | | | |
| KIRC | 16245 | Shorter | 2.61E−07 | 1.58E−07 | 8.61E−08 | 57 |
| | | survival | | | | |
| KIRC | 15406 | Shorter | 3.31E−07 | 1.62E−07 | 6.34E−08 | 29 |
| | | survival | | | | |
| KIRC | 17177 | Shorter | 5.14E−07 | 3.82E−07 | 2.40E−07 | 70 |
| | | survival | | | | |
| HNSC | 17542 | Shorter | 7.32E−06 | 5.14E−05 | 2.36E−05 | 225 |
| | | survival | | | | |
| HNSC | 17364 | Shorter | 1.32E−05 | 6.66E−05 | 3.40E−05 | 219 |
| | | survival | | | | |
| LUSC | 15022 | Longer | 4.19E−05 | 4.21E−05 | 2.39E−05 | 115 |
| | | Survival | | | | |

TABLE 3

| Module | |
|---|---|
| number | Genes |
| 16873 | BRAF, HRAS, NRAS, KRAS, RAF1 |
| 16157 | RAF1, BRAF |
| 15406 | FGFRL1, EHD2, SSFA2, RFPL1, RFPL3, SLC4A1, |
| | ICOSLG, RASL10A, AP2M1, GAS2L1, CDR1, CD86, |
| | PLCH2, NPC1, ICOS, EHBP1, LAMP5, LAMP3, EXOC1, |
| | ICAM4, ANK1, RHAG, RFPL2, TACC1, GAS2, CNTNAP2 |
| 16245 | ZFX, STAR, AKAP10, ZFY, SOX9, MYCBPAP, FDXR, |
| | LIPE, PLIN1, AMH, SRY, NR5A1, PRKAR2B, NPRL2, |
| | CCHCR1, TCP11L2, PHOX2A, GLCE, PHLPP1, PDE8A, |
| | PDE8B, NGDN, HAND2, EPSTI1, EMC8, NPAS3, KDM5D, |
| | CYP11B2, CYP11B1, KDM5C, NR0B1, AKAP13 |
| 13986 | FGFRL1, RASL10A, SSFA2, RFPL1, RFPL3, SLC4A1, |
| | GAS2L1, ICAM4, TACC1, ANK1, RHAG, RFPL2, GAS2 |
| 17177 | CLCA3P, ZFX, ZFY, NGDN, FBP1, FBP2, LYRM4, FDXR, |
| | PLIN1, SRY, NR0B1, DYNLT3, SPTLC3, IMPA1, IMPA2, |
| | THOC7, PDE8A, PDE8B, EPSTI1, KDM5D, KDM5C, STAR, |
| | FUBP3, MYCBPAP, NPRL2, AMH, TCP11L2, SLC5A10, |
| | NR5A1, TRPM4, KLHDC4, GLCE, GCH1, EMC8, |
| | TOMM40L, PRKAR2B, AKAP13, AKAP10, CLCA1, SOX9, |
| | TRAK2, PHLPP1, HS3ST5, IPO11, CKMT1B, HAND2, LIPE, |
| | GCHFR, NPAS3, CPT1A, CCHCR1, ALS2, NIF3L1, |
| | PHOX2A, SSR1, SSR2, CYP11B2, CYP11B1 |
| 17542 | BRCA1, TP53, CDK6, CCND1, CDKN1A, ATM, RB1, CDK3, |
| | CDK4, RAD51, CHEK2 |
| 17364 | BRCA1, TP53, ATM, RAD51, CHEK2 |
| 15022 | ARID1B, MRGBP, MPP6, CSMD2, CSMD3, CSMD1, EP400, |
| | ACTL6A, ARID4A, URI1, MRFAP1 |
| 17542 | BRCA1, TP53, CDK6, CCND1, CDKN1A, ATM, RB1, CDK3, |
| | CDK4, RAD51, CHEK2 |
| 9853 | MYH2, MYH3, MYH1, MYH4, MYH8 |
| 17611 | FYN, HRAS, MAPK14, PTK2B, CRK, RAF1, NRAS, MAPK3, |
| | GRAP2, MAPK7, SRC, SHC1, PRKCE, ILKAP, RASA1, |
| | KRAS, NCK1, SOS1, PTPN11, CBL, GRB2, BRAF, RHO, |
| | MAPK1 |
| 17552 | NRAS, HRAS, PTK2B, CRK, RAF1, FYN, SRC, SHC1, |
| | RASA1, KRAS, NCK1, SOS1, PTPN11, CBL, GRB2, BRAF, |
| | RHO |

“Mapping engine,” as described herein, refers to a software system that is designed to assign attributes to a module on a multiscale module map. Examples of mapping engines include but are not limited to systems which implement mapping functions that take input attributes describing basic molecular elements of a patient or sample such as genes or proteins and module attribute values for one or more modules in the multiscale map. Multiscale maps and methods to make one are indicated in Example 1 (Static multiscale map) and Example 2 (Multidimensional multiscale map). Mapping engines can also implement mapping functions that take one or more module-level attribute and output another module level attribute. A mapping function can determine module attribute values based on the attribute value for neighboring or descending modules in the multiscale map. For instance, module-level mutation scores can be computed by summing mutation events to modules representing individual genes, which are descendants of the module in the multiscale map. A node A descends from the node B if there exists a series of parent-child relations that connects node A to node B. As another example, a binary function can be applied which assigns a value of I to the module when at least one of the descending genes has been observed as mutated in the patient or sample of interest and a value of 0 when none descending genes have been mutated. Arithmetic or geometric mean, median and other summary functions can be used to define module scores based on the descending genes or modules. A scoring function can take into account the hierarchical structure of the multiscale map. For instance module scores can be determined or modified based on the parents or children of the module node in the map. In some embodiments of the method and system, a mapping engine is provided. In the embodiments described herein, the static and multidimensional multiscale maps can be processed using conventional programming codes and software analysis as provided herein and are well known in the art. In the embodiments described herein, the mapping engines can be processed using conventional programming codes as provided herein and are well known in the art.

Techniques described according to some embodiments herein, or portions of these techniques can be implemented in hardware, software, firmware, or combinations thereof. If implemented in software, the techniques can be realized at least in part by a computer-readable medium comprising instructions that, when executed, performs one or more of the methods described above. The computer-readable medium can form part of a computer program product, which can include packaging materials. The computer-readable medium can comprise random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, can be realized at least in part by a computer-readable communication medium that carries or communicates code in the form of instructions or data structures and that can be accessed, read, and/or executed by a computer.

Methods according to some embodiments herein or portions of these methods can be implemented on any conventional host computer system, such as those based on Intel® or AMD® microprocessors and running Microsoft Windows operating systems. Other systems, such as those using the UNIX or LINUX operating system and based on IBM®, DEC® or Motorola® microprocessors are also contemplated. The systems and methods described herein can also be implemented to run on client-server systems and wide-area networks, such as the Internet.

Software to implement a method or model or portion thereof according to some embodiments herein can be written in any well-known computer language, such as, for example, Java, C, C++, Visual Basic, FORTRAN, Python, JavaScript as a programming languages, R, or COBOL and compiled using any well-known compatible compiler. The software according to some embodiments normally runs from instructions stored in a memory on a host computer system. A memory or computer readable medium can be a hard disk, floppy disc, compact disc, DVD, magneto-optical disc, Random Access Memory, Read Only Memory or Flash Memory. The memory or computer readable medium used in accordance with embodiments herein can be contained within a single computer or distributed in a network. A network can be any of a number of conventional network systems known in the art such as a local area network (LAN) or a wide area network (WAN). Client-server environments, database servers and networks that can be used in the accordance with embodiments herein are well known in the art. For example, the database server can run on an operating system such as UNIX, running a relational database management system, a World Wide Web application and a World Wide Web server. Other types of memories and computer readable media are also contemplated to function within the scope of the some embodiments herein. Programming frameworks that can be used can include for example, AngularJS, or Django, or other programming frameworks known to those skilled in the art. Programming libraries that can be used in any of the embodiments described herein can include for example, Sigma.js, Bootstrap, Bioconductor, networkX, or other programming libraries known to those skilled in the art.

The data matrices constructed by the methods or portions thereof according to some embodiments herein can be represented in a markup language format including, for example, Standard Generalized Markup Language (SGML), Hypertext markup language (HTML) or Extensible Markup language (XML). Markup languages can be used to tag the information stored in a database or data structure of some embodiments, thereby providing convenient annotation and transfer of data between databases and data structures. In particular, an XML format can be useful for structuring the data representation of reactions, reactants and their annotations; for exchanging database contents, for example, over a network or internet; for updating individual elements using the document object model; or for providing differential access to multiple users for different information content of a data base or data structure of some embodiments. XML programming methods and editors for writing XML code are known in the art as described, for example, in Ray, Learning XML O′Reilly and Associates, Sebastopol, Calif. (2001).

A computer system according to some embodiments herein can further include a user interface capable of receiving a representation of one or more reactions. A user interface in accordance with some embodiments herein can also be capable of sending at least one command for modifying the data structure, the constraint set or the commands for applying the constraint set to the data representation, or a combination thereof. The interface can be a graphic user interface having graphical means for making selections such as menus or dialog boxes. The interface can be arranged with layered screens accessible by making selections from a main screen. The user interface can provide access to other databases useful in accordance with some embodiments herein, for example an in silico database of patterns of sequence specific labeling of one or more nucleic acids or pluralities thereof, for example host genomes, foreign element genomes, or other extragenomic sequences, or links to other databases having information relevant to the patterns of sequence-specific labeling of various nucleic acids. Also, the user interface can display a graphical representation of a map of patterns or biological process known to those skilled in the art. A map of patterns of labeling, an alignment, or other information in accordance with some embodiments herein, for example, a table, graph, reaction network, flux distribution map or a as a modal matrix. SQL databases, for example, such as Postgres database and noSQL database for instance MongoDB, Hadoop and other databases known to those skilled in the art can be used for any of the embodiments described herein.

In some embodiments, the modules can be compared across multiple patients or samples that can be categorized into different classes or can have different biological or clinical characteristics such as survival, response to treatment, health condition, disease type or subtype or other traits that can be associated with patients or biological samples. In some embodiments, additional module level scores or attributes can be obtained by summarizing the module level scores or attributes within a sample class or condition or time point of interest. In some embodiments a function such as mean, max, min, median, or other summary function can be used to obtain such attributes for modules based on the attributes in the input data. In some embodiments, a statistical test can be used to obtain module level attributes or scores. In some embodiments the statistical test comprises a T-test, cox regression analysis, hypergeometric test, binomial test, and/or a gene set enrichment analysis. In some embodiments, statistical associations are associated with modules in the multidimensional multiscale map.

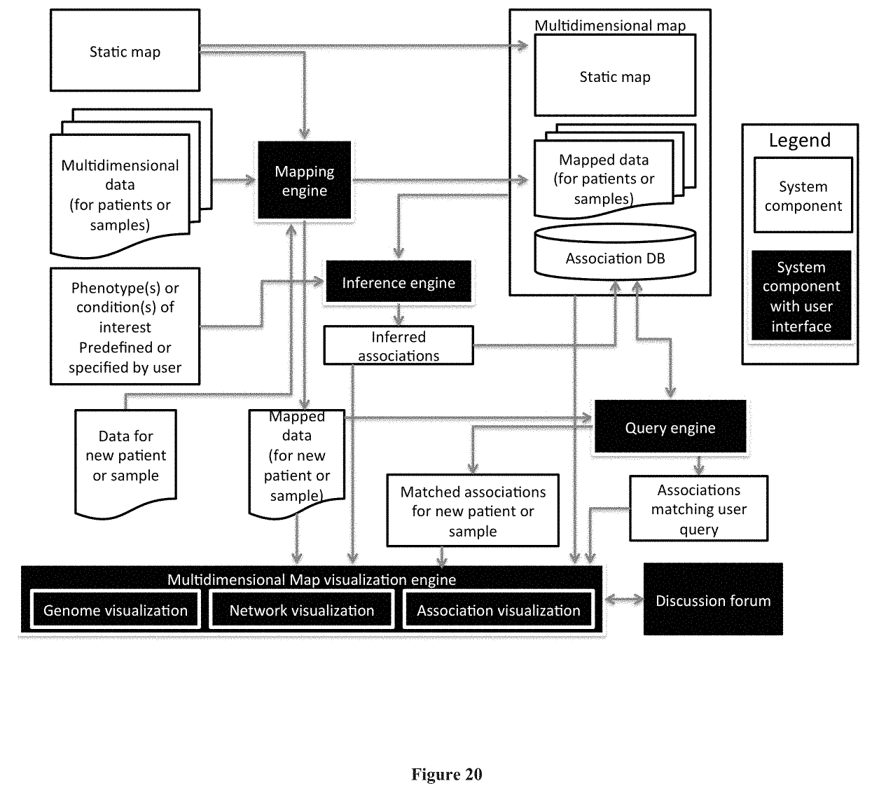

“Query engine” as described herein, refers to a search engine that a user can run in order to identify module associations of interest or identify module associations that match module features of a patient or sample of interest. In some embodiments, a method of making a multidimensional multiscale module map for identifying analyzing and displaying hierarchies of network or pathway activities is provided, wherein the method comprises using a query engine. In some embodiments of the methods and systems described herein, a query engine is provided. Examples of query engines used in the methods described herein can include but are not limited to systems which allow the user to search for associations which include certain specified modules and/or module features and/or values of certain module features. Without being limiting, a query engines can also allow the user to limit the search to associations pertaining to certain biological contexts or associations inferred based on certain biological datasets. A query engine can also allow the user to query for associations that associate certain features of a patient or subject with other biological or clinical features or phenotypes. For example, the query engine can be implemented to query for associations which associate mutations in a gene within a module with patient survival or response to treatment. Examples for using the query engine are shown in FIG. 20 for example.

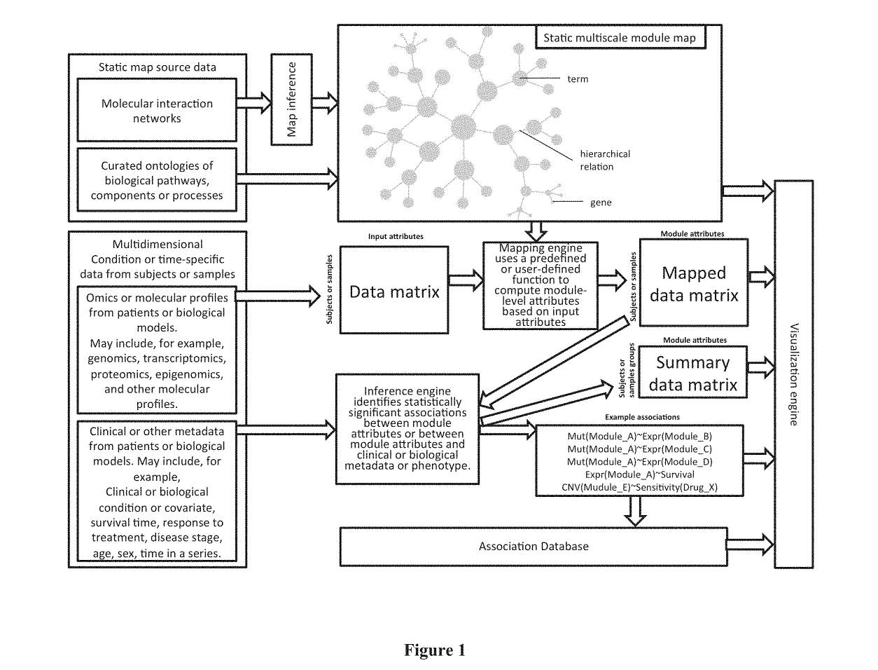

“Visualization engine,” as described herein, refers to a graphic tool for a user implementing any of the methods or systems of any of the embodiments described herein. Examples of how the visualization engine is used is provided in FIGS. 1, 2, and 20, for example. In some embodiments, the visualization engine allows a user to view the multidimensional multiscale module map. In some embodiments, the visualization engine allows a user to visualize the static multiscale module map. In some embodiments, the visualization engine allows a user to visualize the genome of a subject or sample. In some embodiments the visualization engine allows the user to visualize module associations and the data supporting the associations. In some embodiments of the methods and systems described herein, a visualization engine is provided. In the embodiments described herein, the visualization can be processed using conventional programming codes, software analysis and/or processing systems as provided herein. Examples of using the visualization engine are shown in FIGS. 1, 2 and 20, for example.

In some embodiments described herein, a method of making a multidimensional multiscale module map for identifying analyzing and displaying hierarchies of network or pathway activities is provided. In some embodiments, the method can comprise providing a static multiscale module map, accessing a plurality of measured attributes for a plurality of elements from one or more patients and/or biological samples from at least one condition of interest, assigning a plurality of attributes to a plurality of modules in the static multiscale module map, identifying associations from a plurality of attributes for a plurality of modules, ranking the associations based on significance and module size criteria or other user selected criteria, thereby generating a database of ranked most significant associations and storing a database of the ranked most significant associations along with module attributes and the static multiscale module map, thereby generating said multidimensional multiscale module map. In some embodiments, the assigning is performed by a mapping engine. In some embodiments, the mapping engine is coupled to the static multiscale module map.

“Inference engine,” as described herein, refers to a software system that is designed to identify associations between module attributes or between modules attributes and biological or clinical phenotypes or traits. In some embodiments described herein, an inference engine is provided, wherein the inference engine is used to identify significant associations between a plurality of modules. Without being limiting, examples of inference engines can include systems which perform inference of statistical associations between module attributes and/or between module attributes and biological or clinical attributes or phenotypes. As another example, systems for inferring association rules can be used to infer associations between module attributes and/or between module attributes and biological or clinical attributes or phenotypes. Associations can be identified by comparing the values of a module attribute across multiple samples categorized by one of more conditions of interest where a condition can be determined based on the value of another module attribute or by another attribute describing, for instance, a biological or clinical phenotype. Associations can be identified by correlating the module attribute values with a genomic or clinical trait, feature, measurement or observation that can describe another module in the map or another concept not represented in the map such as a clinical or a biological phenotype. In some embodiments of the methods and systems described herein, an inference engine is provided.

For example, an inference engine can implement a hypergeometric or a gene set enrichment test to determine the association between a condition of interest and module mutation status.

An inference engine can infer a significant association between a certain condition and the over-representation of certain events within a module. For example, an inference engine can compare samples across two or more states to identify mutations that occurred in a particular state and then identifying the modules in the map which have a significantly greater mutation frequency then expected at random, given the overall mutation frequency of the given biological condition. Modules that display significantly more somatic mutations in the condition of interest than expected at random can be determined, for example, by using a binomial test or another relevant test known to those skilled in the art.

An inference engine can infer an association between a condition and module activity or attribute, for example, the average gene expression within the module, by comparing module attribute values across samples assigned to two conditions (the condition of interest and a reference condition) using a statistical test such as a t-test or Wilcoxon test or another test used to compare a continuous or categorical variable across two sets of samples known to those skilled in the art.

An inference engine can infer an association between module activity and multiple conditions, for example, by comparing samples across multiple states using a statistical test, such as, for example, an ANOVA test or another test used to compare a continuous or categorical variable across multiple sets of samples known to those skilled in the art.

Module activity or mutation status can be correlated by an inference engine with subject survival, for example, Cox regression analysis or another means for conditional survival analysis known to those skilled in the art.

An inference engine can be used to identify an association between drug sensitivity of a sample and module activity or mutation status. For example, by using a statistical test to compare the IC 50 indexes or other measures of drug sensitivity known to those skilled in the art, for two or more classes of samples determined by the presence or absence of a mutation within a module of interest or determined by a module feature (e.g. high or low activity). A statistical test, such as, for example, a T-test or Wilcoxon test or another test known to those skilled in the art can be used to compare the IC50 scores across two sets of samples. By way of example and not of limitation, an ANOVA analysis can be used to compare such scores over two or more classes of samples.

In some preferred embodiments, the statistical tests implemented by an inference engine can take into account the hierarchical structure of the module map to provide accurate scores and p-values. In some preferred embodiments module scores can be modified according to their size or the scores of their parents or ancestors or children or descendants in the multiscale module map.

It will be known to those skilled in the art that other tests can be preferentially substituted by other statistical tests known to those skilled in the art depending on the nature and distribution of the gene or module scores and the nature of the type of correlations or associations of interests.

It will also be understood that covariates can be included while computing a statistical test to derive module associations. For example, in some clinical applications covariates such as patients' age, gender, drugs or therapies administered to the patient, or overall tumor mutation frequency can be included in the analysis.

In some embodiments described herein, an inference engine is provided for the method of making a multidimensional multiscale module map.

“Biological sample,” as described herein, refers to cells, tissue, blood and any other type of biological matter that can come from a subject of interest, such as for example, a human or a model organism. Without being limiting, such a biological sample can include, but is not limited to a tissue sample, a tumor sample, a cell or a biological fluid, such as ascites fluid.

“Measured attributes for a plurality of elements of a patient” can refer to features of biological data, such as, features of genes, proteins, DNA, RNA, miRNA, epigenetic modifications and variants, and other features of biological data that are known to those skilled in the art. These attributes can all be accessible by omics platforms.

“Cancer,” as described herein refers to malignant tumors, malignant neoplasms, and/or a group of diseases that involve abnormal cell growth that can have the potential to invade or spread to other body parts. There are many types of cancers. Cancer can include but it is not limited to, lung, breast, kidney, colon, bladder, brain, cervical, endometrial, gallbladder, liver, pancreatic, prostate, thyroid, uterine, bone, leukemia, stomach, uterine, ovarian, skin cancer, and other types of cancers that are known to one skilled in the art. In some embodiments, a method of identifying biomarkers of a biological or medical condition of interest is provided. In some embodiments, a method for diagnosing a patient or sample is provided. In some embodiments, the patient has cancer. In some embodiments, the medical condition is cancer. In some embodiments, the cancer is lung, breast, kidney, colon, bladder, brain, cervical, endometrial, gallbladder, liver, pancreatic, prostate, thyroid, uterine, bone, leukemia, stomach, uterine, ovarian, or skin cancer. In some embodiments, the cancer is lung adenocarcinoma, lung squamous cell carcinoma, lung cancer, breast cancer, kidney cancer, or leukemia.

“Biological process” as described herein, refers to processes or mechanisms that occur in a living organism and can include for example, chemical reactions. Biological processes can include but are not limited to, reproduction, digestion, response to stimuli, transcription, translation, cell growth, cell differentiation, morphogenesis, and other processes known to those skilled in the art. In some embodiments described herein, biological processes are pertaining to human biology are included in a static multiscale module map. In some embodiments, the biological process is a mitotic cell cycle, a gene expression pathway, a metabolic pathway, an immune system process, an adaptive immune system process, a GPCR signaling pathway, and/or a signal transduction pathway. Signal transduction pathways can include but are not limited to the MAPK/ERK pathway, cAMP dependent pathways, IP₃/DAG pathways, and other signal transduction pathways known to those skilled in the art. In some embodiments, the signal transduction pathways comprise the MAPK/ERK pathway, cAMP dependent pathways, and/or IP₃/DAG pathways.

“Biological databases” as described herein can refer to manually curated databases or publicly available databases that contain biological information. Biological information can include, but is not limited to molecular interaction data, protein-protein interaction data, genetic data, transcriptional data, protein-DNA interaction data, and/or a combination thereof. Examples of biological databases that are publicly available can include but are not limited to Pathway Commons, Reactome, UCSC genome database and browser, Gene Ontology, KEGG, BioGRID. Many other biological databases are known to those skilled in the art.

“Omics,” as described herein, refers to a field of study in biology ending in -omics. Omics data can include, for example, but is not limited to genomics, cognitive genomics, comparative genomics, functional genomics, metagenomics, personal genomics, epigenomics, lipidomics, proteomics, immunoproteomics, nutriproteomics, proteogenomics, structural genomics, transcriptomics, metabolomics, metabonomics, nutritional genomics, nutrigenomics, toxicogenomics, phychogenomics, and stem cell genomics. Omics studies are known to those skilled in the art. In some embodiments a method of making a multidimensional multiscale module map is contemplated. In some embodiments, the method can include integrating patient omics data and transforming the data into module level scores.

“Genomic data” as described herein, refers to data acquired from computational genomics, epigenomics, metagenomics, pathogenomics, proteomics, whole genome sequencing and other methods known to those skilled in the art. Genomic data can include but is not limited to somatic mutations data, gene copy-number, gene expression data, somatic mutations, gene copy-number, DNA sequences, RNA sequences, and proteomic measurements.

The “cloud based online system,” as described herein refers to a cloud computing system that involves deploying groups of remote servers and software networks that allow a centralized data storage and online access to computer services or resources. Clouds can be classified as public, private or hybrid. In some embodiments, a cloud based system is provided that has a web-browser interface for analyzing a genome sequence from a patient or sample, either the entire genome sequence of an organism or parts of the genome for example the exome of an organism. The user can also use the system to analyze the somatic variants in a tumor genome where the germline variants have been removed either prior to the analysis or during the analysis using specific filters that the invention can implement. The system displays a human reference genome or a genome of another organism. Germline or somatic variants of a subject or sample can be shown around this reference, either over or below the reference genome. A table of germline or somatic variants can be displayed and coordinated with the genome view. The table can contain variant identification information and annotations such as occurrence within general populations or disease populations and other variant annotations or scores, statistics, measurements, p-values, q-values, or other values associated with a variant. The table can be integrated with the genome view. For example, when the user selects a row or variant in the table the genome view is refocused on that variant. Conversely, if the user selects a variant in the genome view, the table is scrolled and refocused to display variant information. In some embodiments, a multidimensional multiscale map is implemented as part of a computer system. In some embodiments, the computer system is a cloud-based online system. In some embodiments, a system for discussing genomic features of a subject or sample is provided. In some embodiments, the system comprises a set of user information embodied in a computer readable medium that represents users that wish to participate in online discussions, a set of instructions for transmitting online discussion between users, an interactive visualization framework with a web-browser interface, a discussion wall, the multidimensional multiscale module map of any one of the embodiments described herein and a static multiscale module map described in any one of the embodiments described herein. A discussion wall as described herein refers to a section of a users' profile where a user and other users can write messages as a public writing space so others that view the profile can see what has been written on the wall.

In some embodiments, the system can be implemented on computer, a server, cluster of computers or servers. In some embodiments, the system described herein can be implemented as an online system or as a local desktop application.

In some embodiments, a method of making a multidimensional multiscale module map for identifying, analyzing and displaying hierarchies of network or pathway activities is provided [FIG. 1, FIG. 20]. In some embodiments, the method can comprise providing a static multiscale module map, accessing a plurality of measured attributes for a plurality of elements from one or more patients and/or biological samples from at least one condition of interest, assigning a plurality of attributes to a plurality of modules in the static multiscale module map, identifying associations from a plurality of attributes for a plurality of modules, thereby generating a database of significant associations and storing the most significant associations along with module attributes and the static multiscale module map in a database, thereby generating said multidimensional multiscale module map. In some embodiments, the multidimensional multiscale map comprises data from patients, cell lines or animal modules that are integrated and transformed into module level scores using a mapping engine. In some embodiments, the modules can be compared across multiple patients or samples that can be categorized into different classes or can have different biological or clinical characteristics such as survival, response to treatment, health condition, disease type or subtype or other traits that can be associated with patients or biological samples. In some embodiments, additional module-level scores or attributes can be obtained by summarizing the module level scores or attributes within a sample class or condition or time point of interest. In some embodiments a function such as mean, max, min, median, or other summary function can be used to obtain such attributes for modules. In some embodiments, a statistical test can be used to obtain module level attributes or scores. In some embodiments the statistical test comprises a T-test, cox regression analysis, hypergeometric test, binomial test, and/or a gene set enrichment analysis. In some embodiments, statistical associations are associated with modules in the multidimensional multiscale map.

The multidimensional multiscale map can have reference points that are indicated by module colors that indicate the change in mutation frequency of genes that are part of a given module. The change can be computed, for example, by comparing samples across two or more states to identify gene mutations that occurred in a particular state, mapping gene level mutation scores to module-level mutation scores and then identifying the modules in the map which have a significantly greater mutation frequency then expected at random, given the overall mutation frequency of the given biological condition. Modules which display significantly more somatic mutations than expected at random can be shown by specific colors that are indicated by a key.

In some embodiments, the multidimensional multiscale map can have reference points that are indicated by module colors that indicate a change in mutation frequency of genes falling within a given module. In some embodiments, a change in mutation frequency can be computed by comparing samples across two or more states to identify mutations that occurred in a particular state and then identifying the modules in the map which have a significantly greater mutation frequency then expected at random, given the overall mutation frequency of the given biological condition.

Multidimensional multiscale map can also be generated to show changes in module activity from one condition to another. The changes can be computed, for example, by comparing module scores or attributes across two states using a statistical test such as a t-test or Wilcoxon test or another test used to compare a continuous or categorical variable across two sets of samples known to those skilled in the art. Modules can be assigned an attribute to denote the aggregated value of another module-level attribute in a condition of interest. The aggregated value can be obtained by taking the mean, median, max, min or another summary function of the module attribute of interest. Modules can be assigned a new attribute based of the significance of the change in another module attribute across two conditions. Values of module attributes can be assigned colors and can be visualized on the map. The color representation of the different colors can be described by a key.

In some embodiments, the modules are assigned an attribute to denote the aggregated value of another module-level attribute in a condition of interest. In some embodiments, the aggregated value is obtained by taking the mean, median, max, min or another summary function of the module attribute of interest. In some embodiments, are assigned a new attribute based on the significance of change in another module attribute across two conditions. In some embodiments, the values of module attributes can be assigned colors and can be visualized on the map. In some embodiments, a key is provided, wherein the color representation of the different colors are described in the key.

In some embodiments, the multiscale map is generated to show changes in module activity from one condition to another. In some embodiments, the changes are computed by comparing samples across two states using a statistical test such as a t-test or Wilcoxon test or another test used to compare a continuous or categorical variable across two sets of samples. In some embodiments, the modules are assigned different colors based on lower or higher average gene expression or another measurement that can be assigned to modules. In some embodiments, the modules are assigned an attribute to denote the aggregated value of another module-level attribute in a condition of interest. In some embodiments, the aggregated value is obtained by taking the mean, median, max, min or another summary function of the module attribute of interest. In some embodiments, the modules are assigned a new attribute based of the significance of the change in another module attribute across two conditions. In some embodiments, the module attributes are assigned a value, wherein the value is assigned a color for visualization on the multidimensional multiscale map.

In some multidimensional multiscale map, module colors can be assigned to indicate an association between an event of a mutation in a gene with a given module and patient survival using for example, a Cox regression analysis.

In some embodiments, a multidimensional multiscale map is provided. In some embodiments, module colors are assigned to indicate an association between an event of a mutation in a gene with a given module and patient survival. In some embodiments, a method of making a multidimensional multiscale module map for identifying analyzing and displaying hierarchies of network or pathway activities. In some embodiments, an analysis is performed with a Cox regression analysis.

In some multidimensional multiscale map, module colors can be assigned to indicate the change in drug sensitivity depending on the mutation status of a given module. The change can be computed, for example, by comparing drug sensitivity for samples with or without mutations within genes belonging to a module. The color representation of the different colors can be described by a key. In some embodiments of making a multidimensional multiscale module map for identifying analyzing and displaying hierarchies of network or pathway activities is provided. In some embodiments, the multidimensional map comprises module colors. In some embodiments, the module colors can be assigned to indicate a change in drug sensitivity depending on the mutation status of a given module. In some embodiments, the change is computed by comparing drug sensitivity for samples with or without mutations within genes belonging to a module. In some embodiments, the multidimensional multiscale map comprises a key. In some embodiments, a color representation of different module colors on the multidimensional multiscale map are described by the key.

In some multidimensional multiscale map, modules are indicated on the map that can be represented by nodes and can be assigned detailed network views which can display relationships between entities belonging to a module. In some embodiments, a multidimensional multiscale module map for identifying analyzing and displaying hierarchies of network or pathway activities is provided. In some embodiments, a multidimensional multiscale module map is provided. In some embodiments, modules are indicated on the multidimensional multiscale map that can be represented by nodes and can be assigned detailed network views which can display relationships between entities belonging to a module.

In some embodiments, a multidimensional multiscale module map for identifying analyzing and displaying hierarchies of network or pathway activities is provided. In some embodiments, the multidimensional multiscale module map for identifying, analyzing and displaying hierarchies of network or pathway activities is generated according to any one of the embodiments described herein. In some embodiments, the multidimensional multiscale module map comprises the static multiscale modular map integrated with patient genomic data to identify modules and pathways altered in a disease. In some embodiments, the genomic data comprises somatic mutations, gene copy-number, and/or gene expression dimensions. In some embodiments, the multidimensional multiscale module map comprises modules. In some embodiments, the modules represent genes, proteins, mRNA, microRNA, amino acid sequences, DNA sequences, protein complexes, cellular components, functions or processes or other biological entities and/or a combination thereof. In some embodiments, the multidimensional multiscale module map comprises associations between modules. In some embodiments, the associations between modules comprises biological and clinical characteristics. In some embodiments, the biological and clinical characteristics comprises survival predictions, response to treatment predictions, health conditions, disease types, disease subtypes and/or other traits associated with patients or biological samples. In some embodiments, the multidimensional multiscale module map comprises at least one identifier for at least one condition of interest. In some embodiments, the at least one condition of interest is a disease and/or cancer.

In embodiments described herein, the multidimensional multiscale map can be implemented as part of a computer system, for instance a web-based or a cloud-based online system. In some embodiments, the multidimensional multiscale map is visualized using an interactive visualization framework with a web-browser interface. In some embodiments, the interface can allow the user to zoom in and out of the selected regions of the map and search for selected biological modules. The user can dynamically select which module scores for which conditions or states are visualized. The system can also provide the ability to search for modules or module relations or module associations in the static or multidimensional multiscale module map. When a search is performed, modules (appearing as nodes in the map) or relations or associations (appearing as edges in the map) matching search criteria can be displayed on a list. After the user selects one of the search results, the map can be focused on the selected node with appropriate zoom values to make the selected node or edge clearly visible to the user. Values assigned to modules or genes or other entities preferably appearing as nodes in the networks can be presented in a table, for example, with one row per node in the network. The table can preferably be integrated with the network to allow automatic focusing on relevant table row or a network node when the user selects a node or table row, respectively. Similarly a table can be implemented where rows preferably correspond to network edges and the table can be integrated with the network to allow automatic refocusing of the table or the network when the user selects an edge or a network row, respectively.

As described herein, a method and system for integrating and analyzing genomic and clinical data to support the identification of new cancer biomarkers and therapeutic targets is provided. In some embodiments, a system for integrating multidimensional patient genomic data (including but not limited to somatic mutations, copy-number and gene expression dimensions) with a multiscale modular map of the cell to identify the common modules and pathways altered in cancer is also provided. In some embodiments, the method automatically identifies robust multi-level genomic module alterations in patient genomes and associates them with clinical and molecular outcomes. In some embodiments, the system can provide a common framework to integrate molecular pathways and networks providing a systems-level model of health or disease conditions or other biological or experimental conditions. In some embodiments, a method to identify disease-associated aberrations in genes, pathways and networks; identify causal relationships between these modules, and between modules and clinical phenotypes, is contemplated.

As described herein, the method and system can implement one or more layers of abstraction. By way of example, and not of limitation, a method and system can implement a multiscale module layer capturing single loci, networks, pathways, and higher-level processes and functions (collectively referred to as modules). It can also implement a data dimension layer capturing the genetic as well as epigenetic, transcriptomic, or proteomic alterations or other biological or clinical dimensions. Associations between these multidimensional modules (e.g. mutations in module A are likely to cause upregulation of module B), and between modules and clinical phenotypes (e.g. upregulation of module B is associated with poor survival, or chemotherapy resistance) can be identified, reported and displayed for visual analysis and interpretation.

The methods provided, as described herein, can provide a means to construct a multi-scale catalog of molecular networks, pathways and processes (collectively modules) containing manually-curated pathway catalogs and ontologies or data-driven ontologies constructed de novo from molecular networks. In some embodiments, the methods can employ techniques that identify a hierarchy of processes and functions directly from high-throughput molecular interaction data. In some embodiments, pathways and processes at all levels of granularity can be captured and represented, from very specific to very broad, high-level processes.

Methods and systems are provided that can further involve integrating modules with patient genomic data to identify module-level activities and aberrations. In some embodiments, the method can provide systematic means for integrating somatic mutations, gene expression and copy-number variation data or other omics data with the module map. In some embodiments, the system can provide systematic means for integrating somatic mutations, gene expression and copy-number variation data with the module atlas can be incorporated. Genomic and transcriptomic aberrations at multiple biological scales (individual loci, pathways, and high-level processes) can be represented. In some embodiments, genomic and transcriptomic aberrations at multiple biological scales are presented. In some embodiments, individual loci, and groups of interacting or functionally related loci including protein complexes, pathways, and biological processes are presented.

In some embodiments, a method of identifying common biological mechanisms in multiple conditions is provided. In some embodiments, the method can comprise identifying of module-module and module-phenotype associations based on multi-dimensional genomic and clinical data. Current bioinformatics platforms provide the ability to identify statistical associations at the gene level (e.g. mutation in gene A is associated with a change in expression of gene B). Recent analyses identified pan-cancer associations between gene mutations and patient survival (Kandoth, C. et al. Nature 502, 333-339 (2013); hereby incoroporated by reference in its entirety). However, due to small sample size the power to detect associations with smaller effect or involving rare mutations is limited. In some embodiments described herein, the method can provide preferable means to systematically identify association at the pathway or network level, providing the opportunity to aggregate weaker effects and increase statistical power (Ideker, T. et al. Cell 144, 860-3 (2011); hereby incorporated by reference in its entirety). In some embodiments, the method is implemented to test for existence of robust statistical associations between multiscale genomic aberrations and downstream molecular and clinical phenotypes (e.g. gene expression and overall patient survival).

A method can be implemented as computer-implemented system that integrates multidimensional cancer genomic data with a multiscale map of networks and pathways to enable complex systems-level inferences.

In an exemplary embodiments, the method provides a means to use a patient genome or other molecular profile to identify facts, annotations, and associations—either literature derived or inferred directly from data—that related to certain features of the patient genome.

In some embodiments, the method provides means for one or more users to identify, comment and share information of biological or clinical interest.

In some embodiments of the system described herein, the system provides means for one or more users to identify, comment and share information of biological or clinical interest.

Genomic or other omics data such as transcriptomic, proteomic, epigenetic or other molecular data can be acquired from patient samples or other biological samples using multiple technologies known to those skilled in the art. For instance, various DNA and RNA sequencing (Shendure, J. et al. Nat. Biotechnol. 26, 1135-1145 (2008), Schadt, E. et al. Annu. Rev. Genomics Hum. Genet. 9, 387-402 (2008), and Ozsolak, F. et al. Nat. Rev. Genet. 12, 87-98 (2011); hereby incorporated by reference in its entirety) array-bases approaches (Auer, H. et al. Methods Mol. Biol. 509, 35-46 (2009); hereby incorporated by reference in its entirety) or proteomics techniques can be applied (Carapito, C. et al. Proteomics 12, 1073-1073 (2012), De Hoog, C. L. et al. Annu. Rev. Genomics Hum. Genet. 5, 267-293 (2004), Hanash, S. et al. Nature 422, 226-232 (2003) and Aebersold, R. et al. Nature 422, 198-207 (2003); all hereby incorporated by reference in their entirety). Such data can be deposited in multiple databases that are also known to those skilled in the art. Those skilled in the art will know that omics data from patients and other biological samples can be downloaded from a variety of databases. These data can further be preprocessed to obtain intensity levels, alterations, or other scores specific to a basic-level biological entity such as a gene or a protein or a miRNA or a DNA or RNA sequence. For instance, sequence alignment (Li, H. et al. Brief. Bioinform. 11, 473-483 (2010), Li, H. et al. Genome Res. 18, 1851-8 (2008), and Richter, B. G. et al. PLoS Comput. Biol. 5, (2009); all hereby incorporated by reference in their entirety), variant calling (Li, H. et al. Genome Res. 18, 1851-8 (2008) and Ji, H. P. et al. Genome Med. 4, 7 (2012); all incorporated by reference in their entirety) and somatic mutation identification tools (Koboldt, D. C. et al. Genome Res. 22, 568-576 (2012), Löwer, M. et al. PLoS Comput. Biol. 8, (2012) and Kim, S. Y. et al. BMC Bioinformatics 14, 189 (2013); all incorporated by reference in their entirety) can be used to identify somatic mutations in genes. RNA processing tools can be used to obtain expression levels for genes as well as other scores, which can be mapped to genes such as alternative splicing events [FIG. 1]. Similarly, protein expression levels, epigenetic changes and other scores can be identified using technologies and tools known to those skilled in the art. Given a set of samples, the scores can be arranged in the form of a two-dimensional matrix where, for instance, rows can represent genes or other basic-level biological entities and columns can represent patients or samples. In such a way a sample-by-gene matrix can be obtained. Clinical parameters or covariates or other samples-specific annotations can also be collected and arranged in a feature-by-patient or a feature-by-sample matrix (Chuang, H.-Y. et al. Mol. Syst. Biol. 3, 140 (2007); incorporated by reference in its entirety). In some embodiments, omics data is provided.

Reference genome for human or other organisms can be applied from databases known to those skilled in the art. For instance human reference genome in one or multiple versions can be downloaded, for example, from the UCSC database (Karolchik, D. et al. Nucleic Acids Res. 42, (2014); incorporated by reference in its entirety).

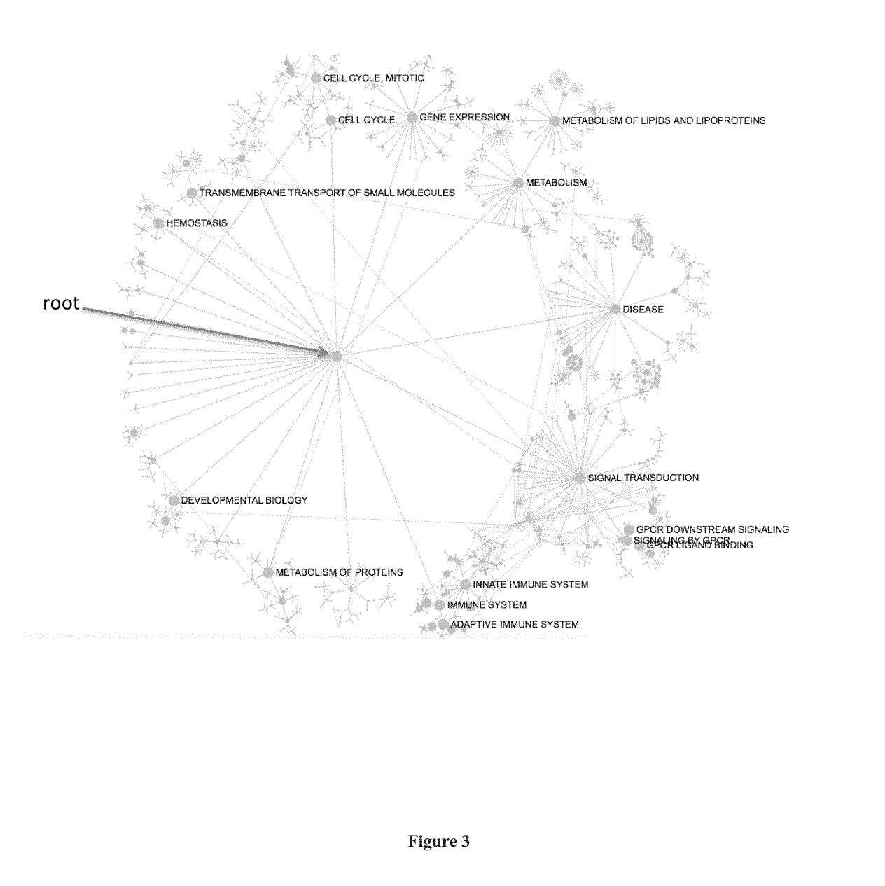

The invention can involve acquiring a hierarchical network representation of the cell [FIG. 3] containing multiple modules which can represent genes, proteins, mRNA, microRNA, amino acid sequences, DNA sequences, protein complexes, cellular components, functions or processes or other biological entities or a combination of the these, where some or all of these entities participate in relations of type (A, B) where A is a member of B, or A is a subtype of B, or A is a subcomponent of B, or A is a specialization of B. In some embodiments, a hierarchical network can be obtained from a manually-curated database. In some embodiments, the manually-curated database could be, for instance, the Gene Ontology database, the Pathway Commons Database, the Reactome Database, or one or more of other databases known to those skilled in the art or a combination of such databases (Ashburner, M. et al. Nat. Genet. 25, 25-29 (2000), Cerami, E. G. et al. Pathway Commons, a web resource for biological pathway data. Nucleic Acids Res. 39, D685-D690 (2011) and Reactome. Genome Biol. 8, R39 (2008); all incorporated by reference in their entirety). Such a hierarchical network will be referred to as a (static) multiscale module map.

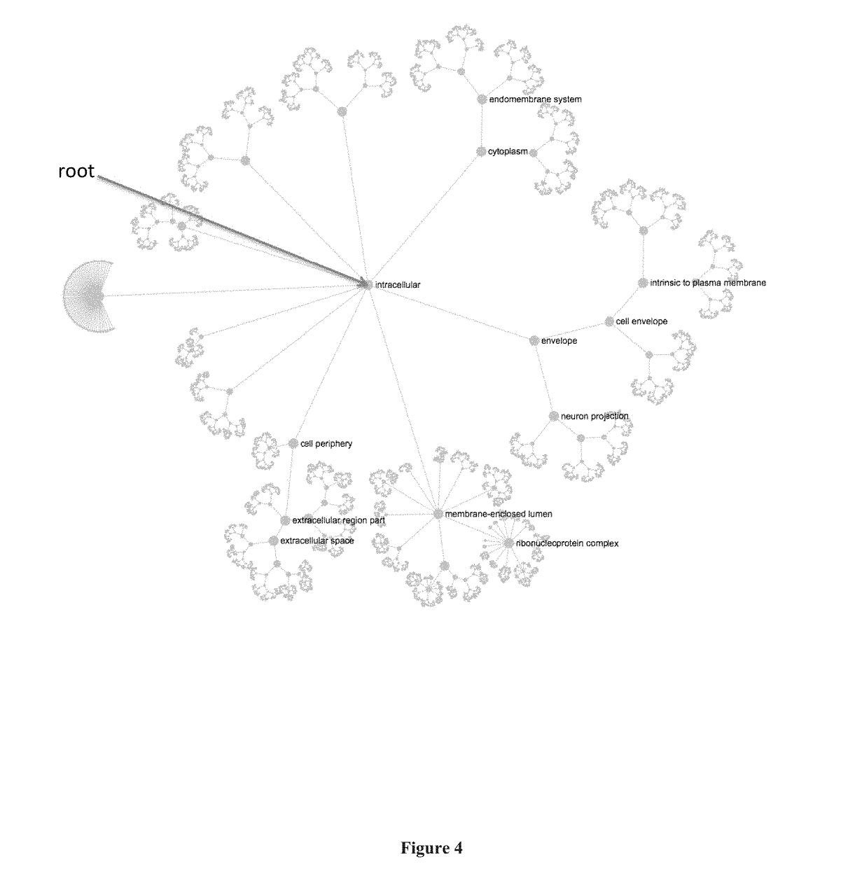

In some embodiments a static multiscale module map can be inferred from data (Dutkowski, J. et al. Nat. Biotechnol. 31, 38-45 (2013); incoporated by reference in its entirety) [FIG. 4]. The inference of a hierarchy for example from molecular interaction data such as, for example, protein-protein, genetic, transcriptional, protein-DNA, or other biological networks or a combination of such networks and can be performed using a method known to those skilled in the art. For instance, it can be performed using a method which iteratively joins the most similar entities to construct a binary clustering tree or dendrogram (Fortunato, S. et al. Phys. Rep. 486, 75-174 (2010) and Oldham, M. C. et al. Proc. Natl. Acad. Sci. U.S.A. 103, 17973-8 (2006); incorporated by reference in their entirety). Various similarity functions can be used in the implementation of the method for example correlation, connectivity, or topological overlap (Fortunato, S. et al. Phys. Rep. 486, 75-174 (2010) and Zhang, B. et al. Stat. Appl. Genet. Mol. Biol. 4, Article 17 (2005); incoporated by reference in its entirety) and others as are known to those skilled in the art. In other embodiments, a multiscale network map can be inferred using a procedure which constructs a binary tree or dendrogram in a way to optimize the probability of the underlying interaction data (Park, Y. et al. BMC Bioinformatics 12, S44 (2011); incoporated by reference in its entirety). A binary tree can first be constructed and further refined to a non-binary tree by subsequent local optimization of the probability score (Dutkowski, J. et al. Nat. Biotechnol. 31, 38-45 (2013); incoporated by reference in its entirety). In other embodiments, a method based on iterative finding of cliques in a network with weighted edges can be applied to construct the static multiscale module map (Kramer, M. et al. Bioinformatics 30, (2014); incorporated by reference in its entirety).

A method for supplementing the clustering tree with new connections from child nodes to additional parents based on the support for such connections in the input network can be used. The tree can hence be transformed into a directed acyclic graph (DAG) (Dutkowski, J. et al. Nat. Biotechnol. 31, 38-45 (2013); incoporated by reference in its entirety).

Module labels and annotations can be mapped from other databases including ontology databases, pathway databases or protein complex databases. These can be mapped directly based on common identifiers, or they can be mapped based on text mining or they can be mapped based on the similarity of the genes sets, for instance based on the Jaccard index, or the similarity of the underlying network topology or a combination of the above methods (Fuxman Bass, J. I. et al. Nat. Methods 10, 1169-76 (2013); hereby incorporated by reference in its entirety herein). An ontology alignment procedure, such as for instance the procedure described in Dutkowski, J. et al. (Nat. Biotechnol. 31, 38-45 (2013); incoporated by reference in its entirety) can be applied to directly compare the multiple multiscale maps, identify common and distinct modules and relations and transfer module annotations.

To determine the most confident modules in a static multidimensional map a measure of module quality can be applied that considers the underlying network support of the module or its robustness to random perturbations of the input data as assessed using a bootstrapping procedure where a portion, for example 5 or 10 percent of the input network data is substituted by random interactions (Dutkowski, J. et al. Nat. Biotechnol. 31, 38-45 (2013); incoporated by reference in its entirety).

Module scores can be determined for modules in the multiscale-network map based on the scores for neighboring or descending entities. For instance, module-level mutation scores can be computed by summing mutation events to individual genes represented by nodes which descend from the module node in the multiscale map. A node A descends from the node B if there exists a series of parent-child relations that connects the node A to the node B. Instead of a sum a binary function can be applied which takes value of 1 when a least one of the descending genes has been mutated of 0 when none descending genes are mutated. Arithmetic or geometric mean, median and other summary functions can be used to define module scores based on the descending genes or modules. A scoring function can take into account the hierarchical structure of the multiscale map. For instance module scores can be determined or modified based on the parents or children of the module node in the map.

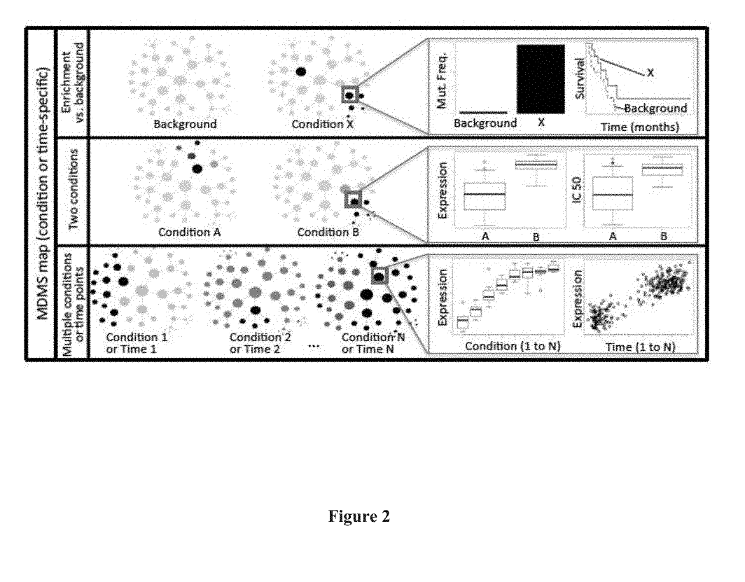

Modules of clinical or biological interest can be identified by comparing dynamic module level activities to identify modules activities which change significantly across conditions of interest or by correlating the module activity with a genomic or clinical trait, feature, measurement or observation that can describe another module in the map or another entity not represented in the map (FIG. 2).

For example, a hypergeometric or a gene set enrichment test can be computed to determine the over-representation of certain events within a module.

Change in mutation frequency of genes falling within a given module can be determined, for example, by comparing samples across two or more states to identify mutations that occurred in a particular state and then identifying the modules in the map which have a significantly greater mutation frequency then expected at random, given the overall mutation frequency of the given biological condition. Modules that display significantly more somatic mutations than expected at random can be determined, for example, by using a binomial test or another relevant test known to those skilled in the art.

Change in module activity, for example, change in the average gene expression, from one condition to another can be computed, for example, by comparing samples across two states using a statistical test such as a t-test or Wilcoxon test or another test used to compare a continuous or categorical variable across two sets of samples known to those skilled in the art.

Change in module activity across multiple conditions can be computed, for example, by comparing samples across two states using a statistical test, such as, for example, an ANOVA test or another test used to compare a continuous or categorical variable across multiple sets of samples known to those skilled in the art.

Module activity or mutation status can be correlated with subject survival using, for example, a cox regression analysis or another means for conditional survival analysis known to those skilled in the art.

Change in drug sensitivity conditional on sample class determined by module activity or mutation status can be computed, for example, by using a statistical test by comparing, for example, the IC 50 indexes or other measures of drug sensitivity known to those skilled in the art, for two or more classes of samples. A statistical test, such as, for example, a T-test or Wilcoxon test or another test known to those skilled in the art can be used to compare the IC50 scores across two sets of samples. By way of example and not of limitation, an ANOVA analysis can be used to compare such scores over two or more classes of samples.

In some preferred embodiments, the statistical tests can take into account the hierarchical structure of the module map to provide accurate scores and p-values. In some preferred embodiments module scores can be modified according to their size or the scores of their parents or ancestors or children or descendants in the multiscale module map.

It will be known to those skilled in the art that other tests can be preferentially substituted by other statistical tests known to those skilled in the art depending on the nature and distribution of the gene or module scores and the nature of the type of correlations or associations of interests.

It will also be understood that covariates can be included while computing a statistical test to derive module associations. For example, in some clinical applications covariates such as patients' age, gender, drugs or therapies administered to the patient, or overall tumor mutation frequency can be included in the analysis.

In a preferred embodiment, the invention provides means to display and browse the list of high-scoring or significant modules and their significant associations with other modules or biological or clinical entities. Preferably, the embodiments described herein provide a way for the user to visualize the data underlying the association. A plot relevant to the statistical test that was applied such as a boxplot, a heatmap, a Kaplan-Meyer plot, scatterplot, a barchart or other plot can be presented to the user, for instance, when the user selects an from the list.

In a preferred embodiment, the methods and systems described herein, provide a means to visualize the multidimensional multiscale map together with the module scores and resulting statistics.

In a preferred embodiment nodes in the multiscale map can be assigned colors to represent statistical scores, p-values, or q-values or other values assigned to a module.

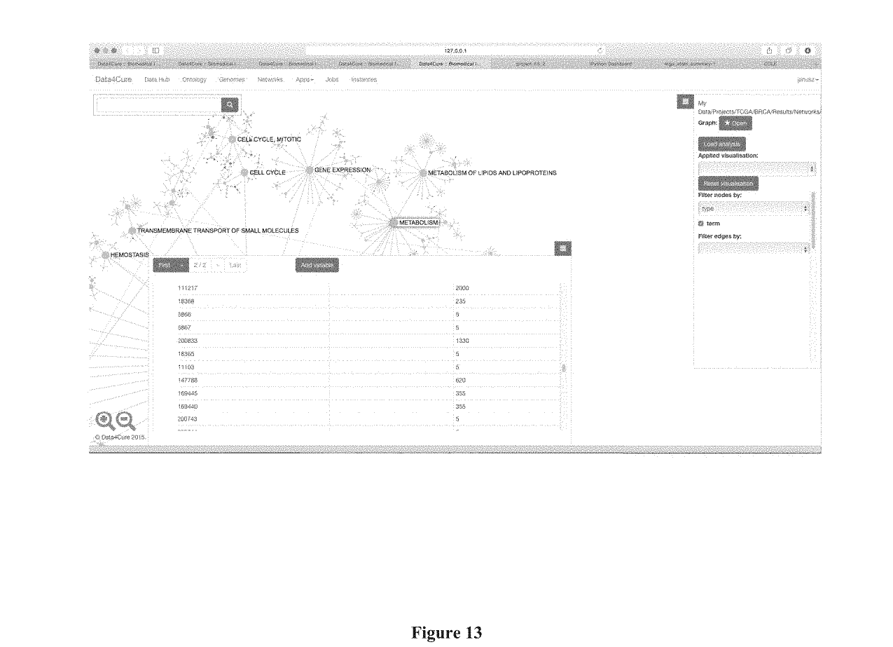

In a preferred embodiment the methods and systems described herein, provide a means to select the visual mapping of the node values to node color, size or other visual parameters. In a preferred embodiment the methods and systems described herein can provide a way to scroll through multiple visual mappings in series so as to visualize the changes in module values from one state or context to another [FIG. 13]. A slider or a list component can be implemented to allow the user to scroll through such a list and select the state or context to be visualized.

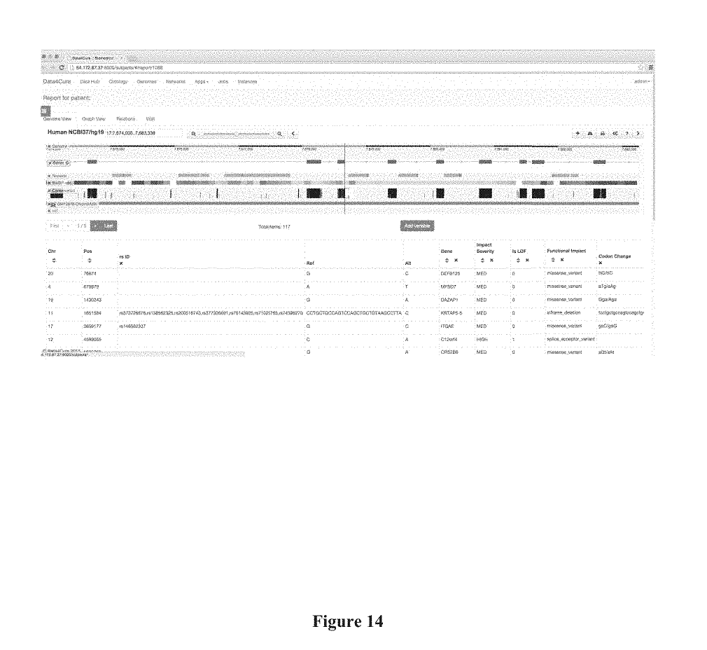

In a preferred embodiment, a table is provided with the method and systems described herein, where the table comprises scores that can be coordinated with the multiscale network map such that when a node is selected in the table the map automatically zooms to that node and when the node is selected in the map the table displays rows with values relevant to the node [FIG. 14]. In a preferred embodiment, plots relevant to the results of a statistical test applied to a node are accessible by the user through a link or a button or a tooltip or a similar device attached to the node in the multiscale map.

In a preferred embodiment, the user can use the methods and systems provided herein, to construct a multiscale map to identify modules of biological or clinical interest in selected biological or clinical conditions. The user can acquire omics or molecular data for the said conditions or for the said conditions and control conditions. The user can also select one of the static multiscale maps that can be provided to the user or the user can provide a multiscale network map or a static multiscale network map can be inferred from molecular interaction networks. The user can then specify a method to compute the module-level matrix based on the basic-level omics or molecular data provided or the user can use one or more of the default methods to perform this task. The user can then choose the method to identify significant or high-scoring module associations for the said conditions. The user can then browse the list of significant or high-scoring modules. The user can also browse the multiscale map on which the said modules are marked by color, size or other visual features. The user can also browse the map with coordinated table of said modules where the table can display the nodes or edges in the map and can also display node or edge properties including module scores, statistics, measurements, p-values, q-values, or other values associated with a node or edge in the map.

In some embodiments, the significant or high-scoring modules can be biomarkers for said conditions. In some embodiments, the significant or high-scoring modules can be biomarkers for patient survival. In some embodiments, the significant or high-scoring modules can be biomarkers for response to treatment.

In some embodiments, the significant or high-scoring modules can provide new drug targets. In some embodiments, the significant or high-scoring modules can provide information of agricultural interest. In some embodiments, the agricultural interest includes biological information of wild type and genetically modified plants. In some embodiments, the significant or high-scoring modules can provide information of clinical or biological interest. In some embodiments, associations between modules can be made to provide biological information of wild type and genetically modified plants. In some embodiments, the associations can show resistance to pests, correlation of diseases, predict environmental conditions, reduction of spoilage, resistance to chemical treatment and/or the nutrient profile of a plant.

In a preferred embodiment, a user can use the multidimensional multiscale map to provide diagnosis or clinical information for a patient. The user can identify or access previously identified significant or high-scoring modules from a multidimensional multiscale map. The user can they obtain omics or molecular data from a patient or a sample of the same type that the data used to construct the multidimensional multiscale map. The user can then obtain the module-level scores for the said patient or the said sample using the method of obtaining module scores that was used to construct the multidimensional multiscale map. The user can then be presented with information from significant or high-scoring modules from the multiscale map with scores matching the patient or sample-specific modules. The said information can be expected patient survival or response to treatment or disease diagnosis or disease subtype diagnosis or other information of clinical or biological interest.

In a preferred embodiment, the user can use the methods and systems described herein to analyze a genome sequence from a patient or samples, either the entire genome sequence of an organism or parts of the genome for example the exome of an organism. The user can also use any one of the methods and/or systems described herein to analyze the somatic variants in a tumor genome where the germline variants have been removed either prior to the analysis or during the analysis using specific filters that the invention can implement.

In a preferred embodiment, the reference genome of an organism is provided, wherein the reference genome can be displayed and the germline or somatic variants of a subject or sample can be shown around this reference, either on top, below or over the reference genome.

In a preferred embodiment, a table or tables are provided. In some embodiments, a table of said germline or somatic variants can be displayed and coordinated with the genome view. In some embodiments, the table contains variant identification, occurrence within general populations or disease populations and/or other variant annotations and information such as scores, statistics, measurements, p-values, q-values, or other values associated with a node or edge in the map. The table can be integrated with the genome view. For example, when the user selected a row or variant in the table the genome view can be refocused on that variant. Conversely, if the user selects a variant in the genome view, the table can be scrolled and refocused to display variant information.

In an exemplary embodiment filters can be implemented to allow the user to identify a subset of interesting variants based on annotations and statistics associated with the variants. The user can be able to select interesting filters and assemble a collection of filters by joining individual filters together by a logical “and” or “or” or a combination of “and” and “or” relations. Once applied, filters can be used by the system to subselect rows in the variant table.

In an exemplary embodiment the variants or the variants which pass a set of filters can be displayed on the multiscale module map. Variants can be displayed as individual nodes connected to genes or other entities which they affect. Variants can also be displayed as changes in color or annotation of other nodes in the map, in particular nodes that correspond to genes or other modules which are preferentially effected by the variants.

In a preferred embodiment an association view can be implemented providing means to display and browse the list of high-scoring or significant associations of variants, genes affected by variants or module affected by variants with other genes, modules or biological or clinical entities or their measurements. In some embodiments, the methods and or systems described herein provides a way for the user to visualize the data underlying the scoring. A plot relevant to the statistical test applied such as, for example, a boxplot, a heatmap, a Kaplan-Meyer plot, scatterplot, a barchart or other plot can be presented to the user. Additional association-level filters can be implemented to allow the user to select relations which meet specific criteria, such as p-value or q-value threshold, statistic value threshold or relate specific variants, genes, modules or other biological or clinical entities.

In a preferred embodiment multiple users can access the system of any of the embodiments described herein, and can have access to their own data or data that is shared by multiple users or user groups.

In some embodiments, a discussion system for discussing genomic features of a subject or sample is provided. In some embodiments, the system is a web-based or a cloud-based online system. In some embodiments, the multidimensional multiscale map is visualized using an interactive visualization framework with a web-browser interface. In some embodiments, the system comprises an interface, wherein the interface allows the user to zoom in and out of the selected regions of the map and search for selected biological modules. In some embodiments, system can also provide the ability to search for modules, hierarchical module relations and/or module associations.

In a preferred embodiment a discussion wall can be implemented in any of the systems and methods described herein. Users can be able to post variants, genes, modules, relations or other biological or clinical features specific to the subject or sample to the subject or sample wall. Text comments can be added with each post and users can reply with text comments. In a preferred embodiment, new posts can be made from a variant table from the association table or from the network table and can relate to one or more rows in the table by clicking on the “Post” button or through another way that can be implemented by those skilled in the art. The post can contain reference to the entity or entities selected from the table and can contain an additional textual comments. In a preferred embodiment, responses to the post can be made either from the wall or directly from the variant table or the association table or the network table from which the original post was made. The system can display in each row the information on the number of posts associated with the entity in the row and can allow either placing new posts or replying to prior posts.

In a preferred embodiment some or all the components including the genome browser, the table, the filters the graph view, the association view, the variant and rule comment facility and the comment wall can be integrated together with a unified interface to provide a dynamic and integrated environment for analysis of one or more genomes or other molecular or clinical data including omics data.

### EXAMPLES

### Methods

### Input Data

The Broad GDAC Firehose provides publicly-available preprocessed TCGA level 3 data and level 4 analyses packaged in a form amenable to immediate algorithmic intake (Marx, V. et al. Nat. Methods 10, 293-297 (2013); incorporated by reference in its entirety). From this resource gene mutations were obtained, copy-number variants and mRNA expression levels (microarray and RNAseq) for human subjects as well as selected clinical data such as overall patient survival and the list of administered oncology drugs for each patient were also obtained. Molecular data (including somatic mutations) as well as drug sensitivity data for 504 cancer cell lines were obtained, deposited and made publicly available by the Sage Bionetworks Synapse platform (Friend, S. et al. Cancer Discov. 2, 658 (2012); hereby incorporated by reference in its entirety).

Molecular interaction networks, pathways, processes and components were obtained from publicly available databases. A human protein-protein interaction network was obtained from BioGRID (Stark, C. et al. Nucleic Acids Res. 39, D698-D704 (2011); incorporated by reference in its entirety), a functional gene interaction network HumanNet (Lee, I. et al. Genome Res. 21, 1109-1121 (2011); incorporated by reference in its entirety) was also obtained. A comprehensive collection of manually curated pathways from Pathway Commons (Cerami, E. G. et al. Nucleic Acids Res. 39, D685-D690 (2011); hereby incorporated by reference in its entirety). Static hierarchical ontologies of cellular components, biological processes and molecular functions from the Gene Ontology and the REACTOME database (Ashburner, M. et al. Nat. Genet. 25, 25-29 (2000), Croft, D. et al. Nucleic Acids Res. 39, D691-D697 (2011), Ashburner, M. et al. Nat. Genet. 25, 25-29 (2000); hereby incorporated by reference in its entirety).

### Example 1

### Preparation of the Static Multiscale Module Maps

Modules from manually curated databases were arranged into hierarchies either by exploiting the hierarchical relationships from the input databases or by identifying pairs of modules for which the set of genes of one module was contained in the set of genes of the other module [FIG. 3]. Such multiscale components and functions are captured by the Gene Ontology (GO) which enlists nested biological structures ranging from single genes to macromolecular complexes up to very broad biological processes with thousands of genes. Hierarchically organized catalogs such as GO are well suited to study complex processes arising from coordinated activities of hundreds of genes which can have many specific subfunctions. Pathway sets and hierarchies were obtained from the Pathway Commons and REACTOME databases.

Apart from the manually curated repositories, large-scale data on gene and protein interactions are an increasingly valuable resource for automatically identifying functional hierarchies (Dutkowski, J. et al. Nat. Biotechnol. 31, 38-45 (2013); hereby incorporated by reference in its entirety). Here a hierarchical community detection procedure was applied based on a Bayesian network model and statistical bootstrapping to identify a robust hierarchy of network components (Dutkowski, J. et al. Nat. Biotechnol. 31, 38-45 (2013); hereby incorporated by reference in its entirety). Each module automatically scored based on its support in data and correspondence to known biology as captured by the GO ontology for human, organizing human genes into 16367 biological modules [FIG. 4].

A heuristic for inferring from data and adding to the map new connections from a child node c to an additional parent node p was applied. Starting from the leaves all node pairs (c, p) were considered such that the number of genes assigned to c was less than the number assigned to p. Node p was identified as an additional parent of c if:
1. Nodes p and c were not already on the same path or children of the same node, and
2. There was a dense pattern of interactions connecting genes assigned to c and genes assigned to p (Density≥0.3; hypergeometric P-value<0.05). The sets of genes associated with p and c together formed a dense cluster.

The final result was a DAG T where leaves correspond to genes and non-leaf nodes corresponded to modules containing more than one gene.

The ontology alignment procedure taking into account both the gene composition and the topology of the underlying DAGs was applied to compare the resulting inferred multiscale map to the map obtained from manually curated repositories such as the Gene Ontology (Dutkowski, J. et al. Nat. Biotechnol. 31, 38-45 (2013); hereby incorporated by reference in its entirety). Names and descriptions of the modules in the manually curated maps were transferred to matching modules in the data-driven map.

The network support NS(m) for a module m was computed as the enrichment for interactions connecting genes assigned to the module (−log(P-value) estimated based on the hypergeometric distribution). The bootstrap score B(m) for module m was calculated by randomly removing 5% of the edges in the input network and reconstructing a new bootstrapped multiscale map and aligning it to the original map.

It will be understood that using the above described procedures with varying inputs and parameters one can obtain one or more static multiscale module maps that can include manually curated or inferred modules and hierarchical relationships between these modules.

A static multiscale map containing biological processes pertaining to human biology was constructed by manual curation of biological processes and pathways form literature. Each node in the graph (filled circle) represents a biological module, e.g. a biological process, a pathway or a gene. Each edge represents a hierarchical parent-child relation between modules, for instance, such that one module is considered a parent of another module or one module contains another module, or one module is a generalization of another module. Each module can have multiple children and multiple parents. A root in the hierarchy does not have any parents. Other nodes are labeled with the corresponding names of biological process or pathways. For display purposes, only the names of selected top-level pathways and processes are shown. Node size can indicate the number of genes or other biological entities assigned to the corresponding modules [FIG. 3].

A static multiscale map containing biological processes pertaining to human biology is constructed by automatic analysis of molecular networks in order to infer their underlying hierarchical structure. Each node in the graph (filled circle) represents a group of genes or proteins which can in turn correspond to a biological module, e.g. a biological process, a pathway or a gene. Each edge represents a parent-child relation between modules, for instance, such that one module is considered a child of another module or one module contains another module, or one module is a generalization of another module. Each module can have multiple children and multiple parents. A root in the hierarchy does not have any parents. Other nodes are labeled with the corresponding names of biological process or pathways. For display purposes, only the names of selected top-level pathways and processes are shown. Node size can indicate the number of genes or other biological entities assigned to the corresponding modules. Node sizes indicate the number of genes assigned to a module [FIG. 4].

### Example 2

### Multidimensional Multiscale Module Maps and Applications

Multidimensional multiscale maps were constructed by integrating static multiscale omics maps with multidimensional condition-specific data from cancer patients, cell lines or model organisms Omics data from patients and cell lines were transformed into module-level scores and integrated with the static multiscale map to create a multidimensional multiscale maps. Statistical associations between modules and between modules and clinical or molecular phenotypes were inferred by comparing module scores across multiple patients or samples that can be categorized into different classes or can have different biological or clinical characteristics such as survival, response to treatment, health condition, disease type or subtype or other traits that can be associated with patients or biological samples [FIGS. 1, 2]. Several examples are given below.

Statistical tests, including T-test, Wilcoxon test, cox regression analysis, hypergeometric test, binomial test, gene set enrichment analysis, were used to compare the multiscale module scores across conditions or sample categories or to correlate them with a biological or clinical trait [FIG. 1].

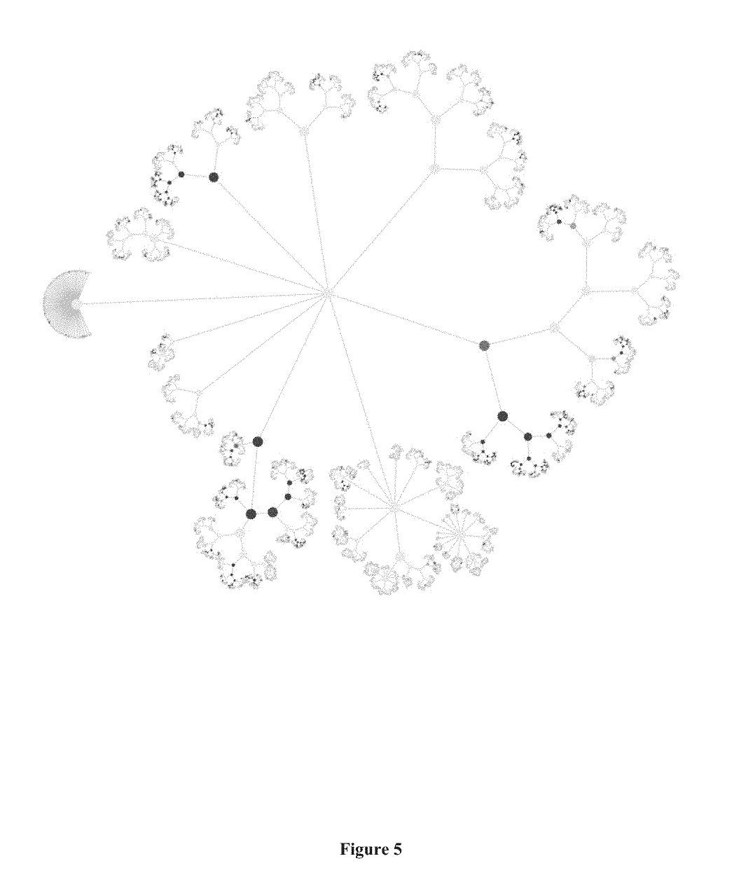

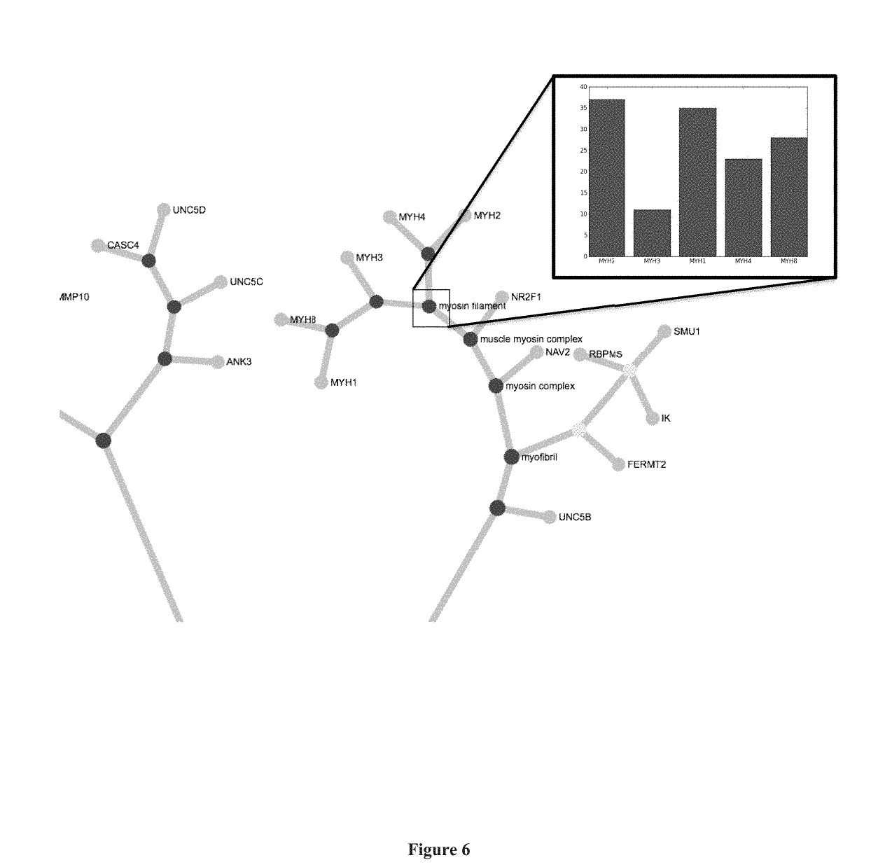

A multidimensional multiscale map was constructed to measure the mutation frequency of genes and modules among 230 lung adenocarcinoma patients from the TCGA cohort [FIG. 5.]. The frequency was computed by comparing samples across two or more states to identify mutations that occurred in a particular state and then identifying the module in the map which have a significantly greater mutation frequency then expected at random, given the overall mutation frequency of the given biological condition. Modules which display significantly more somatic mutations than expected at random in lung adenocarcinomas are shown in darker color. Somatic mutations were determined by comparing lung adenocarcinoma samples with normal samples from the same patients. Part of the map is also shown in FIG. 6. Genes underneath the module 9853 labeled “myosin filament” are frequently mutated in lung adenocarcinomas. The bar chart associated with the selected module shows the number of study cohort patients that have mutations in the genes associated with this module (Table 3 provides a list of genes in this module).

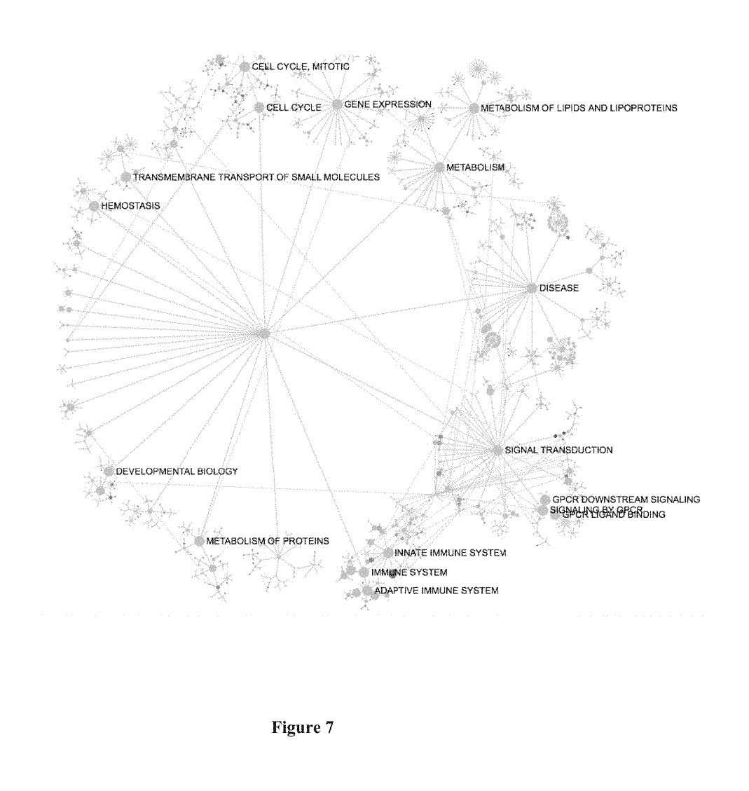

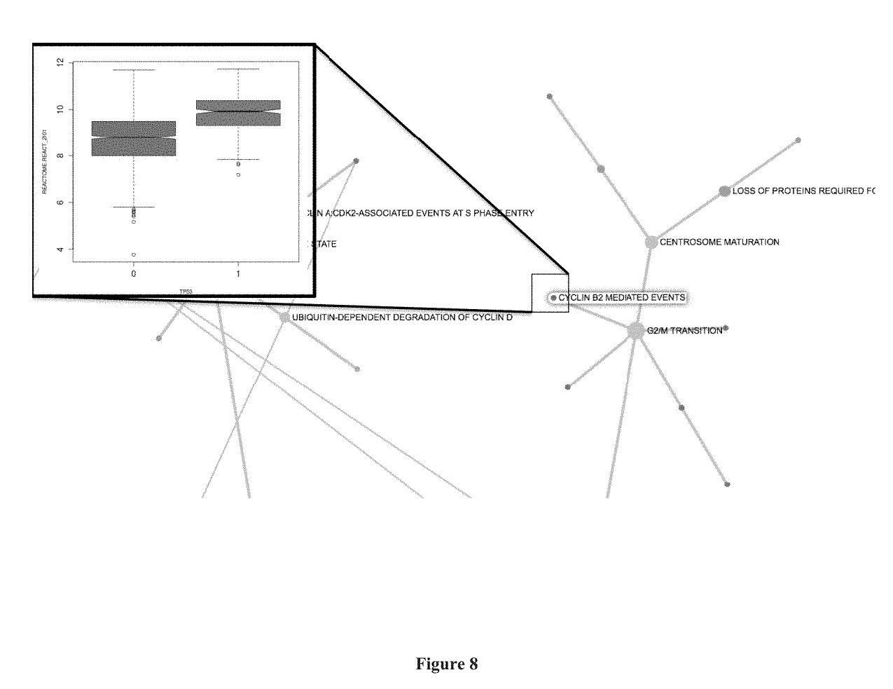

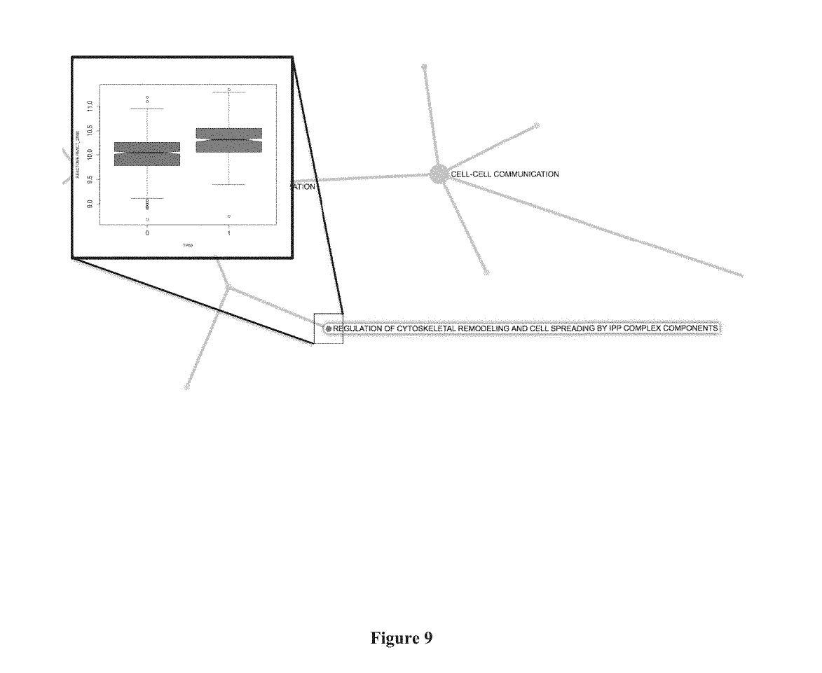

A multidimensional multiscale map was implemented where module colors indicate the change in module activity from one condition to another [FIG. 7]. The change was computed by comparing samples across two states using a t-test. Modules were indicated in darker color if the average gene expression for genes assigned to the module was significantly different in breast cancer samples that harbor a mutation in TP53 gene than in samples with wild-type TP53 gene. Part of the map from FIG. 7 is shown in FIG. 8 focused on module labeled as “Cyclin B2 mediated events”. Genes in this module have higher average gene expression in breast cancer samples that harbor a mutation in TP53 gene than in samples with wild-type TP53. Part of the map from FIG. 7 is shown also in FIG. 9 focused on module labeled as “Regulation of Cytoskeletal Remodeling and cell Spreading by IPP Complex Components”. Genes in this module have higher average gene expression in breast cancer samples that harbor a mutation in TP53 gene than in samples with wild-type TP53. In other examples modules can be assigned different colors based on lower or higher average gene expression or another type of measurement that can be assigned to modules.

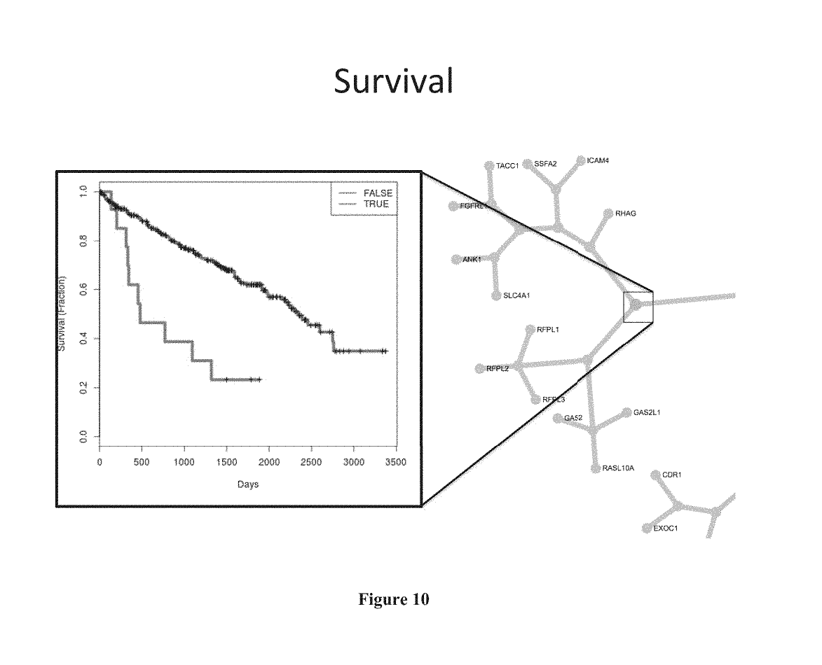

A multidimensional multiscale map was generated where module colors indicate the association between the event of a mutation in a gene within a given module and patient survival in Kidney Renal Clear Cell Carcinoma patients computed using Cox regression analysis [FIG. 10]. Gene mutations in the selected module (13986) are associated with shorter overall survival (P-value=1.2758e-07, Wald test). Such maps were analogously generated for patients with other types of cancer including lung squamous cell carcinoma, and head and neck cancer (head and neck squamous cell carcinoma).

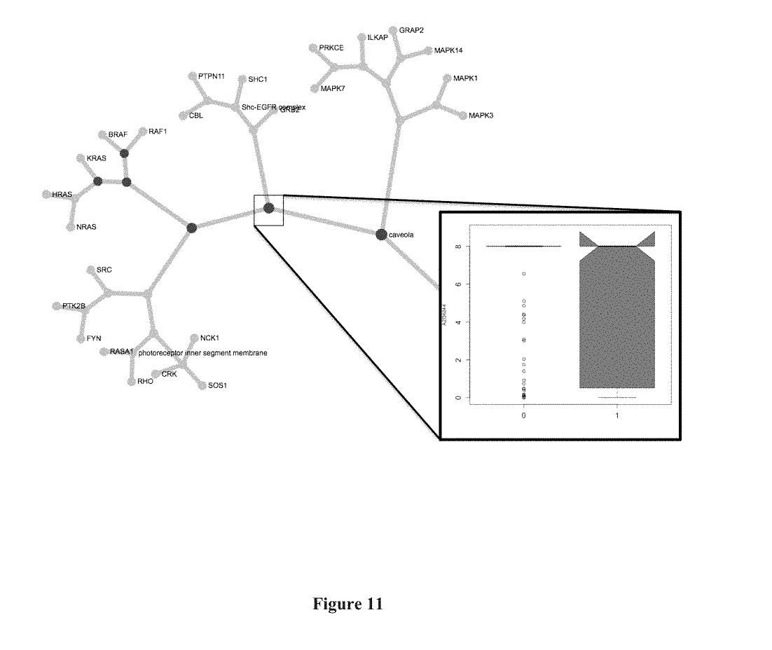

A multidimensional multiscale map was generated where module colors indicate the change in drug sensitivity depending on the mutation status of a given module (i.e. module can contain a mutation in one of the genes or not) [FIG. 11]. The change is computed by comparing drug sensitivity for samples with or without mutations within genes belonging to a module. Drug sensitivity was measured by the IC 50 index (Soothill, J. S. et al. J. Antimicrob. Chemother. 29, 137-139 (1992); hereby incorporated by reference in its entirety). A T-test was used to compare the IC50 scores across two sets of samples. Here mutations in genes belonging to the selected module (module number 17454) are associated with increased sensitivity to the AZD6244 compound (P-value <1.0e-16). Cell lines with a relevant mutation are indicated as “1” in the boxplot and tend to have lower IC 50 values indicating higher sensitivity to AZD6244.

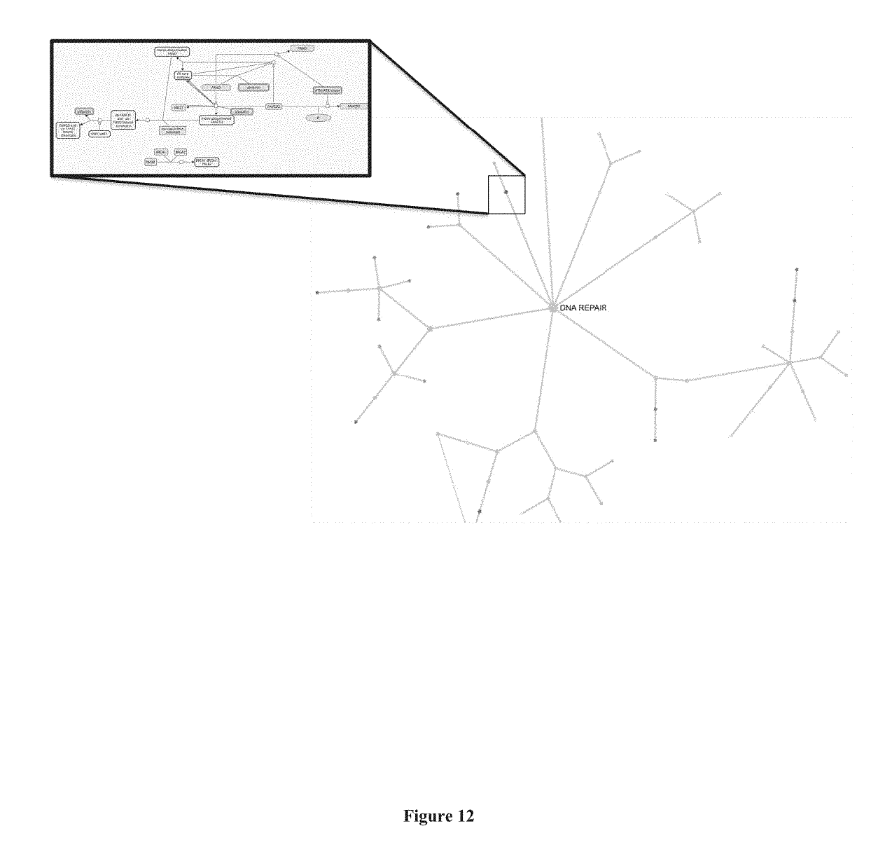

Modules represented by nodes in the multidimensional multiscale map can be assigned detailed network views which can display relationships between entities belonging to the module. An example of a detailed view for the module REACTOME:Fanconi Anemia Pathway is shown in FIG. 12.

A multidimensional multiscale map was implemented as part of a cloud-based online system using an interactive visualization framework with a web-browser interface [FIG. 13]. The interface allows the user to zoom in and out of the selected regions of the map and search for selected biological modules. The user can dynamically select which module scores for which conditions or states to visualize. The system can also provide the ability to search for nodes or edges in the network. When a search is performed, nodes or edges matching search criteria can be displayed on a list. After the user selects one of the nodes or edges in the list the network can be focused on the selected node with appropriate zoom values to make the selected node or edge clearly visible to the user. Values assigned to modules or genes or other entities preferably appearing as nodes in the networks can be presented in a table, for example with one row per node in the network. The table can preferably be integrated with the network to allow automatic focusing on relevant table row or a network node when the user selects a node or table row, respectively. Similarly a table can be implemented where rows preferably correspond to network edges and the table can be integrated with the network to allow automatic refocusing of the table or the network when the user selects an edge or a network row, respectively.

A cloud-based online system with a web-browser interface was implemented for analyzing a genome sequence from a patient or sample, either the entire genome sequence of an organism or parts of the genome for example the exome of an organism. The user can also use the invention to analyze the somatic variants in a tumor genome where the germline variants have been removed either prior to the analysis or during the analysis using specific filters that the invention can implement [FIG. 14]. The system displays a human reference genome or a genome of another organism. Germline or somatic variants of a subject or sample can be shown around this reference, either over or below the reference genome.

A table of germline or somatic variants is displayed and coordinated with the genome view [FIG 14]. The table contains variant identification, occurrence within general populations or disease populations and other variant annotations and information such as scores, statistics, measurements, p-values, q-values, or other values associated with a node or edge in the map. The table can be integrated with the genome view. For example, when the user selected a row or variant in the table the genome view is refocused on that variant. Conversely, if the user selects a variant in the genome view, the table is scrolled and refocused to display variant information.

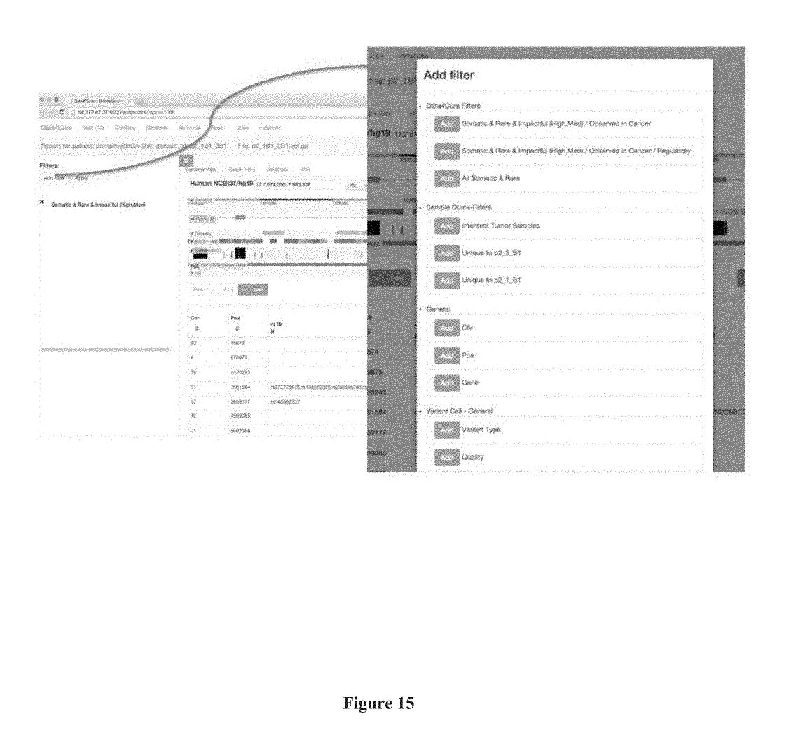

Filters were implemented to allow the user to identify a subset of interesting variants based on annotations and statistics associated with the variants [FIG. 15]. The user is able to select interesting filters from a list of available filters and enable them. Filters are used to subselect rows in the variant table.

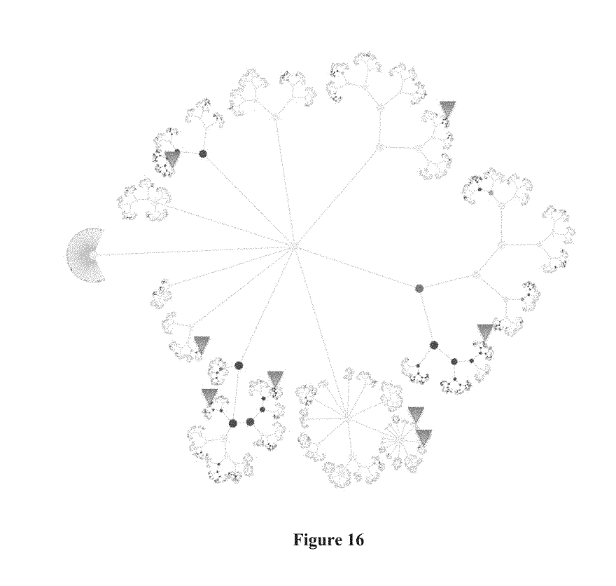

Filters are also used to select variants which are displayed on the multiscale module map [FIG. 16]. Variants can be displayed as individual nodes connected to genes or other entities which they affect. Variants can also be displayed as changes in color or annotation of other nodes in the map, in particular nodes that correspond to genes or other modules which are preferentially effected by the variants.

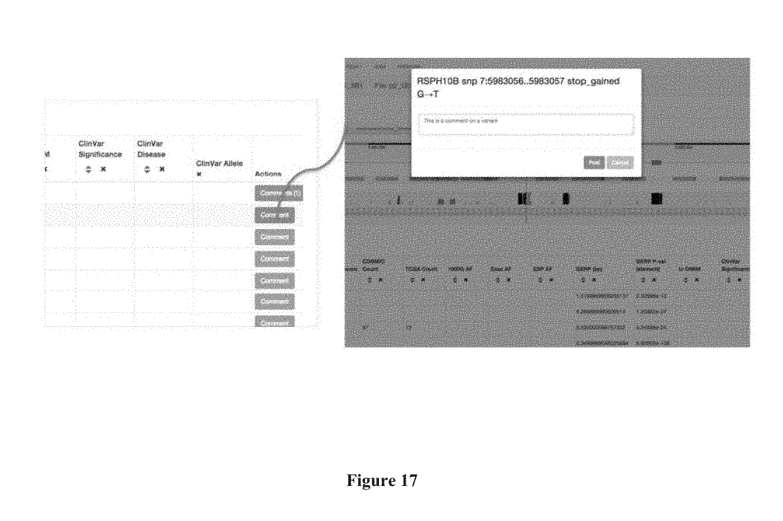

A association view was implemented to provide means to display and browse the list of high-scoring or significant associations of variants, genes affected by variants or modules affected by variants with other genes, modules or biological or clinical entities or their measurements [FIG. 17]. The system allows the user to visualize the data underlying the scoring when an association is selected. A plot relevant to the statistical test applied such as a boxplot, a heatmap, a Kaplan-Meyer plot, scatterplot, a barchart is presented.

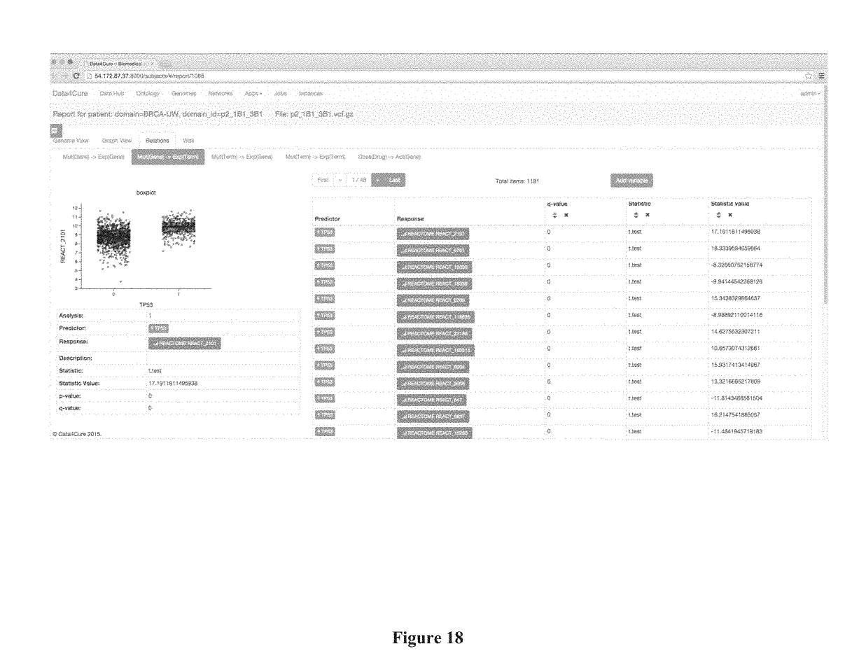

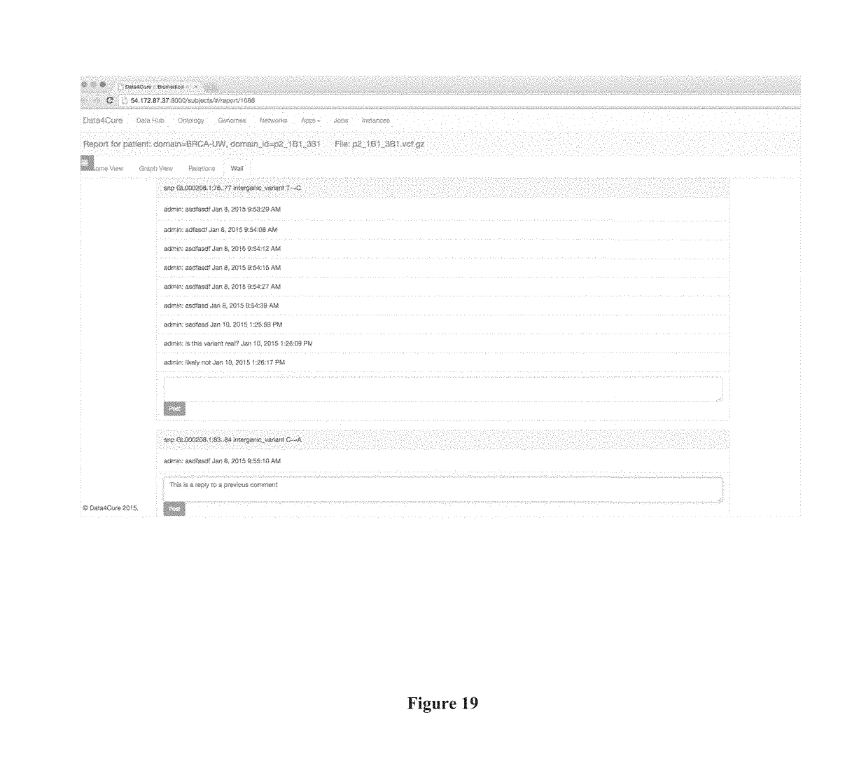

Multiple users who can have access to their own data or data that is shared by multiple users or user groups can access the system. A discussion wall was implemented to allow users to post and discuss variants, genes, modules, relations or other biological or clinical features specific to the subject or sample [FIG. 18]. Text comments can be added with each post and users can reply with text comments. New posts can be made by clicking on the “Post” button from a row in the variant table or the association table or the network table. The post contains a reference to the selected entity from the table and can contain an additional textual comment. Responses to the post can be made either from the wall or directly from the variant table or the association table or the network table from which the original post was made [FIG. 19]. The system displays in each row the information on the number of posts associated with the entity in the row and allows either placing new posts or replying to prior posts.

In the implemented example system all the components including the genome browser, the table, the filters the graph view, the association view, the variant and rule comment facility and the comment wall are integrated together with a unified interface to provide a dynamic and integrated environment for analysis of one or more genomes or other molecular or clinical data including omics data.

### Additional Embodiments

In some embodiments, a method of making a multidimensional multiscale module map for identifying analyzing and displaying hierarchies of network or pathway activities is provided, wherein the method comprises providing a static multiscale module map, accessing a plurality of measured attributes for a plurality of elements from one or more patients and/or biological samples from at least one condition of interest, assigning a plurality of attributes to a plurality of modules in the static multiscale module map, identifying associations from a plurality of attributes for a plurality of modules, and storing a database of the most significant associations along with module attributes and the static multiscale module map, thereby generating said multidimensional multiscale module map. In some embodiments, the method further comprises providing a query engine. In some embodiments, the method further comprises providing a visualization engine. In some embodiments, the method further comprises ranking the associations, thereby generating a database of ranked most significant associations. In some embodiments, the database of associations is ranked by significance and/or module size criteria or other user-selected criteria. In some embodiments, the assigning is performed by a mapping engine. In some embodiments, the mapping engine is coupled to the static multiscale module map. In some embodiments, the identifying is performed by an inference engine. In some embodiments, the static multiscale module map contains biological processes pertaining to human biology. In some embodiments, the biological process is a mitotic cell cycle, a gene expression pathway, a metabolic pathway, an immune system process, an adaptive immune system process, a GPCR signaling pathway, and/or a signal transduction pathway. In some embodiments, the signal transduction pathways comprise the MAPK/ERK pathway, cAMP dependent pathways, and/or IP₃/DAG pathways In some embodiments, the static multiscale module map is constructed by manual curation of biological processes and pathways from literature. In some embodiments, the static multiscale module map is constructed from pathways and processes from biological databases. In some embodiments, the biological databases are Pathway Commons, Gene Ontology, and/or Reactome databases. In some embodiments, the map is constructed by automatic analysis of molecular networks to infer underlying hierarchical structure. In some embodiments, the static multiscale module map is inferred by biological data. In some embodiments, the biological data includes molecular interaction data, protein-protein interaction data, genetic data, transcriptional data, protein-DNA interaction data, and/or a combination thereof. In some embodiments, the biological data represents at least one group of genes and/or proteins that correspond to a biological process. In some embodiments, the static multiscale module map is constructed from iterative findings of cliques in a network. In some embodiments, the static multiscale module map is constructed from manually curated databases arranged into hierarchies by exploiting the hierarchical relationships from input biological databases or by identifying pairs of modules for at least one set of genes of at least one module. In some embodiments, the plurality of measured attributes for a plurality of elements from one or more patients comprises omics data from patients and/or omics data from cell lines. In some embodiments, the omics data comprises data from genomics, cognitive genomics, functional genomics, metagenomics, epigenomics, lipidomics, proteomics, immunoproteomics, proteogenomics, structural genomics, transcriptomics, pharmacogenomics, toxicogenomics, stem cell genomics and/or metabolomics. In some embodiments, the multidimensional multiscale module map comprises the static multiscale modular map integrated with patient genomic data with to identify common modules and pathways altered in a disease. In some embodiments, the genomic data comprises somatic mutations, gene copy-number, gene expression data, somatic mutations, gene copy-number, DNA sequences, RNA sequences, and/or proteomic measurements. In some embodiments, the multidimensional multiscale module map comprises modules. In some embodiments, the multidimensional multiscale module map further comprises associations between modules and biological phenotypes and/or clinical phenotypes, thereby generating inferred associations on the multidimensional multiscale module map. In some embodiments, the modules represent genes, proteins, mRNA, microRNA, amino acid sequences, DNA sequences, protein complexes, cellular components, functions or processes or other biological entities and/or a combination thereof. In some embodiments, the multidimensional multiscale module map comprises associations between modules. In some embodiments, the associations between modules comprises biological and clinical characteristics. In some embodiments, the biological and clinical characteristics comprises survival predictions, response to treatment predictions, health conditions, disease types, disease subtypes and/or other traits associated with patients or biological samples. In some embodiments, the patient suffers from cancer. In some embodiments, the cancer is lung adenocarcinoma, lung squamous cell carcinoma, lung cancer, breast cancer, kidney cancer, or leukemia. In some embodiments, the multidimensional multiscale module map comprises at least one identifier for at least one condition of interest. In some embodiments, the at least one condition of interest is a disease and/or cancer. In some embodiments, the cancer is lung adenocarcinoma, lung squamous cell carcinoma, lung cancer, breast cancer, kidney cancer, or leukemia. In some embodiments, the modules are assigned an attribute to denote the aggregated value of another module-level attribute in a condition of interest. In some embodiments, the aggregated value is obtained by taking the mean, median, max, min or another summary function of the module attribute of interest. In some embodiments, are assigned a new attribute based on the significance of change in another module attribute across two conditions. In some embodiments, the values of module attributes can be assigned colors and can be visualized on the map. In some embodiments, a key is provided, wherein the color representation of the different colors are described in the key.

In some embodiments, a multidimensional multiscale module map for identifying analyzing and displaying hierarchies of network or pathway activities is provided. In some embodiments, the multidimensional multiscale module map for identifying analyzing and displaying hierarchies of network or pathway activities is generated by any of the embodiments described herein.

In some embodiments, a method for graphically displaying information and data from a generated multidimensional multiscale module map is provided. In some embodiments, the method comprises providing the generated multidimensional multiscale module map for identifying analyzing and displaying hierarchies of network or pathway activities of any one of the embodiments described herein, generating a color map from the generated multidimensional multiscale module map and displaying the color map on a screen of a computer device or system. In some embodiments, the method further comprises displaying modules of interest. In some embodiments, the method further comprises displaying hierarchical relationships between modules. In some embodiments, the method further comprises displaying associations between module characteristics and between modules and phenotype characteristics. In some embodiments, the modules are assigned an attribute to denote the aggregated value of another module-level attribute in a condition of interest. In some embodiments, the aggregated value is obtained by taking the mean, median, max, min or another summary function of the module attribute of interest. In some embodiments, are assigned a new attribute based on the significance of change in another module attribute across two conditions. In some embodiments, the values of module attributes can be assigned colors and can be visualized on the map. In some embodiments, a key is provided, wherein the color representation of the different colors are described in the key.

In some embodiments, a method for identifying multiscale biomarkers of a biological or medical condition of interest is provided. In some embodiments, the method comprises providing the generated multidimensional multiscale module map of any of the embodiments described herein for identifying analyzing and displaying hierarchies of network or pathway activities, selecting at least two conditions from the multidimensional multiscale module map, wherein at least one of the conditions is a condition of interest and another condition is a reference condition and querying the map for significant associations for the condition of interest using a query system. In some embodiments, the condition is a disease or cancer. In some embodiments, the cancer is lung squamous cell carcinoma, lung cancer, breast cancer, kidney cancer, or leukemia.

In some embodiments, a method for identifying common biological mechanism in multiple conditions of interest is provided. In some embodiments, the method comprises providing the multidimensional multiscale module map of any of the embodiments described herein, inferring the multiscale biomarkers for each condition of interest, wherein the multiscale biomarkers are identified by any one of the methods of any one of the embodiments described herein. In some embodiments, the condition of interest is a disease or cancer. In some embodiments, the cancer is lung adenocarcinoma, lung squamous cell carcinoma, lung cancer, breast cancer, kidney cancer, or leukemia.

In some embodiments, a method for diagnosing a patient or sample is provided, wherein the method comprises providing the multidimensional multiscale module map of any of the embodiments described herein, wherein the multidimensional multiscale module map comprises inferred associations for the conditions of interest, assigning a plurality of sample attributes to a plurality of modules in the multidimensional multiscale module map, automatically querying the multidimensional multiscale module map for associations matching the one or more mapped module attributes in the query patient or biological sample, and generating a list of the identified associations ranked based on significance and/or module size criteria or other user selected criteria. In some embodiments, the user selected criteria comprises significance threshold, type of association of interest, statistical test used, and/or datasets used to derive the association. In some embodiments, the assigning is performed by a mapping engine. In some embodiments, the mapping engine is coupled to a static multiscale module map.

In some embodiments, a system for discussing genomic features of a subject or sample is provided, wherein the system comprises a set of user information embodied in a computer readable medium that represents users that wish to participate in online discussions, a set of instructions for transmitting online discussion between users, an interactive visualization framework with a web-browser interface, and the multidimensional multiscale module map of any of the embodiments described herein. In some embodiments, the multidimensional multiscale module map is obtained from a cloud based online system. In some embodiments, the system can be implemented on a single computer, a server, a cluster of computers and/or servers.

In some embodiments, a method for providing a group forum for discussing genomic features of a subject or sample and potential treatment options is provided, the method comprising providing the system of any one of any of the embodiments described herein and transmitting online discussions between users. In some embodiments, the genomic features are potential biomarkers. In some embodiments, the genomic features are drug targets. In some embodiments, the genomic features are matched with statistical associations stored in a database.

In some embodiments, a method for providing a group forum for discussing genomic features of a subject or sample and potential treatment options is provided, wherein the method comprises providing the system of any one of the embodiments described herein and transmitting online discussions between users. In some embodiments, the genomic features are potential biomarkers. In some embodiments, the genomic features are drug targets. In some embodiments, the genomic features are matched with statistical associations stored in a database.

In some embodiments, a method for identifying multiscale biomarkers of a biological or medical condition of interest is provided, wherein the method comprises providing the generated multidimensional multiscale module map of any of the embodiments described herein for identifying analyzing and displaying hierarchies of network or pathway activities, selecting at least two conditions from the multidimensional multiscale module map, wherein at least one of the conditions is a condition of interest and another condition is a reference condition, and querying the map for significant associations for the condition of interest using an inference engine and storing a database of the most significant associations, thereby generating a database of identified multiscale biomarkers. In some embodiments, the condition is a disease or cancer. In some embodiments, the cancer is lung adenocarcinoma, lung squamous cell carcinoma, lung cancer, breast cancer, kidney cancer, or leukemia.

In some embodiments, a system for discussing modules and module relations in a static multiscale module map is provided, wherein the system comprises a set of user information embodied in a computer readable medium that represents users that wish to participate in online discussions, a set of instructions for transmitting online discussion between users, an interactive visualization framework with a web-browser interface and a static multiscale module map. In some embodiments, the static multiscale map is constructed by manual curation of biological processes and pathways from literature. In some embodiments, the static multiscale map is constructed from pathways and processes from biological databases. In some embodiments, the biological databases are Pathway Commons, Gene Ontology, and/or Reactome databases. In some embodiments, the static multiscale module map is constructed by automatic analysis of molecular networks to infer underlying hierarchical structure. In some embodiments, the static multiscale module map is inferred from biological data. In some embodiments, the biological data includes molecular interaction data, protein-protein interaction data, genetic data, transcriptional data, protein-DNA interaction data, and/or a combination thereof. In some embodiments, the biological data represents at least one group of genes and/or proteins that correspond to a biological process. In some embodiments, the static multiscale module map is inferred from iterative findings of cliques in a molecular interaction network. In some embodiments, the static multiscale module map is constructed from manually curated databases arranged into hierarchies by exploiting the hierarchical relationships from input biological databases or by identifying pairs of modules for at least one set of genes of at least one module. In some embodiments, the static multiscale module map is obtained from a cloud based online system. In some embodiments, the system can be implemented on a single computer, a server, a cluster of computers and/or servers.

In some embodiments, a system for discussing modules, modules relations, module attributes or activities and module associations in the multidimensional map is provided, wherein the system comprises a set of user information embodied in a computer readable medium that represents users that wish to participate in online discussions, a set of instructions for transmitting online discussion between users, an interactive visualization framework with a web-browser interface and the multidimensional multiscale module map of any one of the embodiments described herein. In some embodiments, the multidimensional multiscale module map is obtained from a cloud based online system. In some embodiments, the system can be implemented on a single computer, a server, a cluster of computers and/or servers.

In some embodiments of the methods, systems and multidimensional multiscale module maps, the embodiments can be used to visualize one or more module attributes on the multidimensional multiscale map. In some embodiments the user can choose which attribute or attributes to visualize. In some embodiments the attributes can provide information about module values of an individual patient or sample. In some embodiments, the attributes can provide information about aggregated module values of a patient or sample group. In some embodiment the group can be a cancer. In some embodiments, the group can be a cancer subtype.

In some embodiments of the methods, systems and multidimensional multiscale module maps, the embodiments can be used to provide means to analyze a genome of a patient or sample in the context of a multidimensional multiscale map. In some embodiments, the visualization engine implemented by the invention can display a filtered list of genomic variants from the patient or sample genome on the multidimensional multiscale map [FIG. 16]. In some embodiments, the module features in the multidimensional multiscale can provide information about the number of mutations in the population that occur within the genes assigned to each module. In some embodiments, the module features in the multidimensional multiscale can provide information about the significance of the number of mutations in the population that occur within the genes assigned to each module. In some embodiments, the population can be a population of disease or cancer patients. When analyzing a genome of a patient or subject using one possible embodiment of the invention the user can be presented with information about mutations occurring within genes that reside within the same modules as the genes mutated in the patient or sample. In some embodiments, the user can be presented with associations that pertain to the modules harboring mutated genes in the sample or patient.

In some embodiments, the associations of modules or module features can specify a list of values for a feature for which the association applies. In some embodiments the associations of modules or module features can specify a range of values for a feature for which the association applies. In some embodiments, these values can be used by the query engine to identify associations that match a user query. In some embodiments, these values can be used by the query engine to identify associations that match the value or values of a module feature or features in a patient or a sample.

In some embodiments, the methods and systems described herein can be used to identify associations based on the value of a module feature. In some embodiments, the methods and systems described herein can be used to identify associations based on the value of a continuous module feature. In some embodiments, the continuous feature can be expression level of a gene or protein or the aggregated expression of a group of genes or protein in a module. In some embodiments, the methods and systems described herein can be used to identify associations based on the value of a discrete module feature. In some embodiments, the discrete feature can express the mutation status of a gene, for example either mutated or wild type, or the presence of a mutation within a group of genes in a module, either that a mutation is present in at least one gene in the module or not. In some embodiments, the module feature and its value used to identify the associations can come from a patient or sample for which module values were determined. In some embodiments, the mapping engine can be used to determine the module attribute values based on the values of input attributes. In some embodiments, the query engine can be used to identify the associations based on the module attribute values as shown in FIG. 20.

1. A method of making a multidimensional multiscale module map for identifying analyzing and displaying hierarchies of network or pathway activities, the method comprising:
providing a static multiscale module map;
accessing a plurality of measured attributes for a plurality of elements from one or more patients and/or biological samples from at least one condition of interest;
assigning a plurality of attributes to a plurality of modules in the static multiscale module map;
identifying associations from a plurality of attributes for a plurality of modules; and
storing a database of the most significant associations along with module attributes and the static multiscale module map, thereby generating said multidimensional multiscale module map.

2. The method of claim 1, wherein the assigning is performed by a mapping engine.

3. The method of claim 2, wherein the mapping engine is coupled to the static multiscale module map.

4. The method of claim 1, wherein the identifying is performed by an inference engine.

5-9. (canceled)

10. The method of claim 1, wherein the static multiscale module map is constructed by automatic analysis of molecular networks to infer underlying hierarchical structure.

11-15. (canceled)

16. The method of claim 1, wherein the plurality of measured attributes for a plurality of elements from one or more patients comprises omics data from patients and/or omics data from cell lines.

17. (canceled)

18. The method of claim 1, wherein the multidimensional multiscale module map comprises an integrated network of patient genomic data with the static multiscale modular map to identify common modules and pathways altered in a disease.

19-23. (canceled)

24. The method of claim 1, wherein the multidimensional multiscale module map further comprises associations between modules and biological phenotypes and/or clinical phenotypes. thereby generating inferred associations on the multidimensional multiscale module map.

25. The method of claim 24, wherein the biological and clinical characteristics comprises survival predictions, response to treatment predictions, health conditions, disease types, and/or other traits associated with patients or biological samples.

26-31. (canceled)

32. A method for graphically displaying information and data from a generated multidimensional multiscale module map, the method comprising:
providing the generated multidimensional multiscale module map for identifying analyzing and displaying hierarchies of network or pathway activities claim 1;
generating a color map from the generated multidimensional multiscale module map and
displaying the color map on a screen of a computer device or system, and/or displaying modules of interest, and/or
displaying hierarchical relationships between modules, and/or
displaying associations between module activities and between module activities and phenotype characteristics, and/or a set of user information embodied in a computer readable medium that represents users that wish to exchange information about modules in the map, and/or a set of instructions for transmitting information between users, and/or an interactive visualization framework with a web-browser interface.

33-35. (canceled)

36. A method for identifying multiscale biomarkers of a biological or medical condition of interest, the method comprising
providing the generated multidimensional multiscale module map of claim 1, for identifying analyzing and displaying hierarchies of network or pathway activities;
selecting at least two conditions from the multidimensional multiscale module map, wherein at least one of the conditions is a condition of interest and another condition is a reference condition; and
querying the multidimensional multiscale module map for significant associations for the condition of interest using a query system and/or
providing the generated multidimensional multiscale module map of claim 1, for identifying analyzing and displaying hierarchies of network or pathway activities; selecting at least two conditions from the multidimensional multiscale module map, wherein at least one of the conditions is a condition of interest and another condition is a reference condition;
querying the multidimensional multiscale module map for significant associations for the condition of interest using an inference engine; and
storing a database of the most significant associations, thereby generating a database of identified multiscale biomarkers.

37-44. (canceled)

45. A method for diagnosing a patient or sample, the method comprising:
providing the multidimensional multiscale module map of claim 1, wherein the
multidimensional multiscale module map comprises inferred associations for the conditions of interest;
assigning a plurality of sample attributes to a plurality of modules in the multidimensional multiscale module map;
automatically querying the multidimensional multiscale module map for associations matching the one or more mapped module attributes in the query patient or biological sample; and
generating a list of the identified associations ranked based on significance and/or module size criteria or other user selected criteria.

46. The method of claim 45, wherein the user selected criteria comprises significance threshold, type of association of interest, statistical test used, and or datasets used to derive the association.

47. The method of claim 45, wherein the assigning is performed by a mapping engine.

48. The method of claim 47, wherein the mapping engine is coupled to a static or a multidimensional multiscale module map.

49-71. (canceled)
